(19) United States
(12) Patent Application Publication
(10) Pub. No.: US 2016/0166565 A1
(43) Pub. Date: Jun. 16, 2016

(54) TREATMENT OF MULTIPLE SCLEROSIS WITH COMBINATION OF LAQUINIMOD AND FLUPIRTINE

(71) Applicants: David Silver, Netanya (IL); Einat Amit Romach, Rehovot (IL)

(72) Inventors: David Silver, Netanya (IL); Einat Amit Romach, Rehovot (IL)

(73) Assignee: Teva Pharmaceutical Industries, Ltd., Petach-Tikva (IL)

(21) Appl. No.: 15/043,244

(22) Filed: Feb. 12, 2016

Related U.S. Application Data
(63) Continuation of application No. 14/447,394, filed on Jul. 30, 2014, now abandoned.
(60) Provisional application No. 61/860,504, filed on Jul. 31, 2013.

(51) Int. Cl. A61K 31/4704; A61K 45/06; B65D 81/26; A61K 9/20; B65D 1/02; B65D 75/36; A61K 31/44; A61K 9/28

(52) U.S. Cl. CPC A61K 31/4704; A61K 31/44; A61K 45/06; A61K 9/2866; A61K 9/2018; A61K 9/2013; A61K 9/2009; A61K 9/2077; B65D 1/0215; B65D 75/36; B65D 81/266

(57) ABSTRACT

This invention provides a method of treating a subject afflicted with multiple sclerosis or presenting a clinically isolated syndrome comprising administering to the subject laquinimod as an add-on therapy to or in combination with flupirtine. This invention also provides a package and a pharmaceutical composition comprising laquinimod and flupirtine for treating a subject afflicted with multiple sclerosis or presenting a clinically isolated syndrome. This invention also provides laquinimod for use as an add-on therapy or in combination with flupirtine in treating a subject afflicted with multiple sclerosis or presenting a clinically isolated syndrome. This invention further provides use of laquinimod and flupirtine in the preparation of a combination for treating a subject afflicted with multiple sclerosis or presenting a clinically isolated syndrome.

This application claims benefit of U.S. Provisional Application No. 61/860,504, filed Jul. 31, 2013, the entire content of which is hereby incorporated by reference herein.

Throughout this application, various publications are referred to by first author and year of publication. Full citations for these publications are presented in a References section immediately before the claims. Disclosures of the documents and publications referred to herein are hereby incorporated in their entireties by reference into this application.

### BACKGROUND

Multiple Sclerosis (MS) is a neurological disease affecting more than 1 million people worldwide. It is the most common cause of neurological disability in young and middle-aged adults and has a major physical, psychological, social and financial impact on subjects and their families, friends and bodies responsible for health care (EMEA Guideline, 2006).

A clinically isolated syndrome (CIS) is a single monosymptomatic attack suggestive of MS, such as optic neuritis, brain stem symptoms, and partial myelitis. Patients with CIS that experience a second clinical attack are generally considered to have clinically definite multiple sclerosis (CDMS). Various MS disease stages and/or types are described in Multiple Sclerosis Therapeutics (Duntiz, 1999). Among them, relapsing-remitting multiple sclerosis (RRMS) is the most common form at the time of initial diagnosis. Many subjects with RRMS have an initial relapsing-remitting course for 5-15 years, which then advances into the secondary progressive MS (SPMS) disease course. There are currently a number of disease-modifying medications approved for use in relapsing MS (RMS), which includes RRMS and SPMS (The Disease Modifying Drug Brochure, 2006). These include interferon beta 1-a (Avonex® and Rebif®), interferon beta 1-b (Betaseron®), glatiramer acetate (Copaxone®), mitoxantrone (Novantrone®), natalizumab (Tysabri®) and Fingolimod (Gilenya®). Immunosuppressants or cytotoxic agents are used in some subjects after failure of conventional therapies. However, the relationship between changes of the immune response induced by these agents and the clinical efficacy in MS is far from settled (EMEA Guideline, 2006).

Other therapeutic approaches include symptomatic treatment which refers to all therapies applied to improve the symptoms caused by the disease (EMEA Guideline, 2006) and treatment of acute relapses with corticosteroids. While steroids do not affect the course of MS over time, they can reduce the duration and severity of attacks in some subjects.

### Flupirtine

Flupirtine is a centrally acting, non-opioid analgesic. For many years, flupirtine and its physiologically tolerated salts have been successfully used in the therapy of e.g. neuralgias, pain due to degenerative joint diseases, headaches and postoperative pain. U.S. Pat. No. 5,284,861 discloses the use of flupirtine to treat all disorders which are associated with muscular tension (rigidity) and their sequelae, such as for example neuralgias, arthritis, arthrosis, chronic or episodic tension headache, postoperative disabilities, generalized tendomyopathies, insertion tendopathies, Parkinsonian disorders (in particular the rigidity accompanying Parkinsonian disorders).

According to DE 41 22 166 A1, flupirtine can also be used as a medication to treat disorders or disorder symptoms due to muscle tension or resulting from such muscle tension. In U.S. Pat. No. 5,721,258 the use of flupirtine in the treatment of cerebral ischemia and neurodegenerative diseases including multiple sclerosis, Huntington's disease and Alzheimer's disease is further described. From DE 195 41 405 A1, the use of flupirtine in the prophylaxis and therapy of diseases that are accompanied by an impaired haematopoetic cell system is known. Further, DE 100 48 969 A1 describes the use of flupirtine in the treatment of tinnitus. The production of flupirtine and its physiologically usable salts are described in DE 17 95 858 C2, DE 31 33 519 C2, DE 34 16 609 A1 and PCT International Application Publication No. WO 2011/157719, which are hereby incorporated by reference into this application.

Flupirtine was used in a clinical trial in RRMS patients (Clinical Trials Website, article entitled “Flupirtine as Oral Treatment in Multiple Sclerosis (FLORIMS)” retrieved on Jul. 23, 2013 from ClinicalTrials.gov, <http://clinicaltrials.gov/ct2/show/NCT00623415>). The flupirtine dose used in this trial was 300 mg daily (divided in two doses).

Flupirtine is mainly applied orally. DE 93 21 574 U1 describes, for example, pharmaceutical formulations in the form of tablets, granules or pellets containing flupirtine maleate as an active ingredient. DE 43 19 649 A1, U.S. Application Publication No. 2008-0279930 and U.S. Pat. No. 6,194,000 disclose solid flupirtine-containing oral dosage forms with controlled release of the active ingredient. The patent application DE 34 16 609 A1 describes pharmaceutical formulations in the form of injectable flupirtine-gluconate-solutions produced using suitable solvents. PCT International Application Publication No. WO 2011/157719 discloses injectable dosage forms of flupirtine including lyophilisate comprising flupirtine salt and cyclodextrins or cyclodextrin derivatives. PCT International Application Publication No. WO 2004/0112754 A1 discloses a lyophilisate containing the active ingredient flupirtine in its base form or as a physiologically tolerated salt, which may be used to produce a pharmaceutical composition to be parenterally applied.

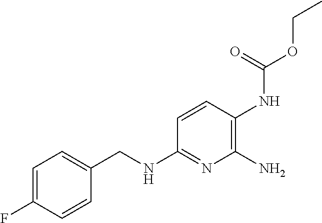 [SMILES: CCOC(=O)Nc1ccc(NCc2ccc(F)cc2)nc1N]

IUPAC: ethyl {2-amino-6-[(4-fluorobenzyl)amino]pyridin-3-yl}carbamate

Flupirtine maleate is available under the trademarks Katadolon S-Long® or Trancolong® (extended release formulations) and Katadolon® (immediate release). An injectable product is sold under the name Katadolon Inject®.

According to the European Medicine Agency (http://www.ema.europa.eu/ema/index.jsp?curl=pages/medicines/human/referrals/Flupirtine-containing medicines/human referral prac 000019.jsp&mid=WC0b01ac05805c516f, retrieved Jul. 23, 2013), flupirtine is available as 100-mg immediate-release capsules, 400-mg extended-release tablets, 75-mg and 150-mg suppositories and as a solution for injection (100 mg).

### Laquinimod

Laquinimod (TV-5600) is a novel synthetic compound with high oral bioavailability which has been suggested as an oral formulation for the treatment of Multiple Sclerosis (MS) (Polman, 2005; Sandberg-Wollheim, 2005; Comi et al 2008). Laquinimod and its sodium salt form are described, for example, in U.S. Pat. No. 6,077,851. The mechanism of action of laquinimod is not fully understood.

Animal studies show it causes a Th1 (T helper 1 cell, produces pro-inflammatory cytokines) to Th2 (T helper 2 cell, produces anti-inflammatory cytokines) shift with an anti-inflammatory profile (Yang, 2004; Brück, 2011). Another study demonstrated (mainly via the NFkB pathway) that laquinimod induced suppression of genes related to antigen presentation and corresponding inflammatory pathways (Gurevich, 2010). Other suggested potential mechanisms of action include inhibition of leukocyte migration into the CNS, increase of axonal integrity, modulation of cytokine production, and increase in levels of brain-derived neurotrophic factor (BDNF) (Runström, 2006; Brück, 2011).

Laquinimod showed a favorable safety and tolerability profile in two phase III trials (Results of Phase III BRAVO Trial Reinforce Unique Profile of Laquinimod for Multiple Sclerosis Treatment; Teva Pharma, Active Biotech Post Positive Laquinimod Phase 3 ALLEGRO Results).

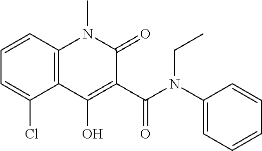 [SMILES: CCN(C(=O)c1c(O)c2c(Cl)cccc2n(C)c1=O)c1ccccc1]

IUPAC: 5-chloro-N-ethyl-4-hydroxy-1-methyl-2-oxo-N-phenyl-1,2-dihydroquinoline-3-carboxamide

### Combination Therapy

The administration of two drugs to treat a given condition, such as multiple sclerosis, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, 1999). In one example, combined administration of fingolimod and interferon (IFN) has been experimentally shown to abrogate the clinical effectiveness of either therapy. (Brod 2000) In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry, 1999). The interaction may also heighten or lessen the side effects of each drug. Hence, upon administration of two drugs to treat a disease, it is unpredictable what change will occur in the negative side profile of each drug. In one example, the combination of natalizumab and interferon β-1a was observed to increase the risk of unanticipated side effects. (Vollmer, 2008; Rudick 2006; Kleinschmidt-DeMasters, 2005; Langer-Gould 2005)

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs (Guidance for Industry, 1999).

Therefore, the state of the art at the time of filing is that the effects of combination therapy of two drugs, in particular laquinimod and flupirtine, cannot be predicted until the results of a combination study are available.

### BRIEF DESCRIPTION OF THE DRAWINGS

FIG. 1 is a graphical representation of the experimental results from Example 1. The graph shows the clinical score for the EAE rodents in each group (on the y-axis) against the days after induction of the disease (on the x-axis).

### SUMMARY OF THE INVENTION

The subject invention provides a method of treating a subject afflicted with multiple sclerosis or presenting a clinically isolated syndrome comprising administering to the subject an amount of laquinimod and an amount of flupirtine.

The subject invention also provides a package comprising: a) a first pharmaceutical composition comprising an amount of laquinimod and a pharmaceutically acceptable carrier; b) a second pharmaceutical composition comprising an amount of flupirtine and a pharmaceutically acceptable carrier; and c) instructions for use of the first and second pharmaceutical compositions together to treat a subject afflicted with multiple sclerosis or presenting a clinically isolated syndrome.

The subject invention also provides laquinimod for use as an add-on therapy or in combination with flupirtine in treating a subject afflicted with multiple sclerosis or presenting a clinically isolated syndrome.

The subject invention also provides flupirtine for use as an add-on therapy or in combination with laquinimod in treating a subject afflicted with multiple sclerosis or presenting a clinically isolated syndrome.

The subject invention also provides a pharmaceutical composition comprising an amount of laquinimod and an amount of flupirtine for use in treating a subject afflicted with multiple sclerosis or presenting a clinically isolated syndrome, wherein the laquinimod and the flupirtine are prepared to be administered simultaneously, contemporaneously or concomitantly.

The subject invention also provides a pharmaceutical composition comprising an amount of laquinimod and an amount of flupirtine.

The subject invention also provides use of an amount of laquinimod and an amount of flupirtine in the preparation of a combination for treating a subject afflicted with multiple sclerosis or presenting a clinically isolated syndrome wherein the laquinimod and the flupirtine are prepared to be administered simultaneously, contemporaneously or concomitantly.

The subject invention also provides a pharmaceutical composition comprising an amount of laquinimod for use in treating a subject afflicted with multiple sclerosis or presenting a clinically isolated syndrome as an add-on therapy or in combination with flupirtine.

The subject invention also provides a pharmaceutical composition comprising an amount of flupirtine for use treating a subject afflicted with multiple sclerosis or presenting a clinically isolated syndrome as an add-on therapy or in combination with laquinimod.

The subject invention also provides a pharmaceutical composition comprising an amount of laquinimod for use in treating a subject afflicted with multiple sclerosis or presenting a clinically isolated syndrome simultaneously, contemporaneously or concomitantly with flupirtine.

The subject invention also provides a pharmaceutical composition comprising an amount of flupirtine for use treating a subject afflicted with multiple sclerosis or presenting a clinically isolated syndrome simultaneously, contemporaneously or concomitantly with laquinimod.

The subject invention also provides a therapeutic package for dispensing to, or for use in dispensing to, a subject afflicted with multiple sclerosis or presenting a clinically isolated syndrome, which comprises: a) one or more unit doses, each such unit dose comprising: i) an amount of laquinimod and ii) an amount of flupirtine, wherein the respective amounts of said laquinimod and said flupirtine in said unit dose are effective, upon concomitant administration to said subject, to treat the subject, and b) a finished pharmaceutical container therefor, said container containing said unit dose or unit doses, said container further containing or comprising labeling directing the use of said package in the treatment of said subject.

The subject invention also provides a pharmaceutical composition in unit dosage form, useful in treating a subject afflicted with multiple sclerosis or presenting a clinically isolated syndrome, which comprises: a) an amount of laquinimod; b) an amount of flupirtine, wherein the respective amounts of said laquinimod and said flupirtine in said composition are effective, upon concomitant administration to said subject of one or more of said unit dosage forms of said composition, to treat the subject.

### DETAILED DESCRIPTION OF THE INVENTION

The subject invention provides a method of treating a subject afflicted with multiple sclerosis or presenting a clinically isolated syndrome comprising administering to the subject an amount of laquinimod and an amount of flupirtine.

In an embodiment of the present invention the method comprises periodically administering to the subject an amount of laquinimod and an amount of flupirtine, wherein the amounts when taken together are effective to treat the subject. In another embodiment, the amount of laquinimod and the amount of flupirtine when administered together is more effective to treat the subject than when each agent at the same amount is administered alone.

In an embodiment of the present invention, the multiple sclerosis is relapsing multiple sclerosis. In another embodiment, the relapsing multiple sclerosis is relapsing-remitting multiple sclerosis.

In one embodiment, the amount of laquinimod and the amount of flupirtine when taken together is effective to reduce a symptom of multiple sclerosis in the subject. In another embodiment, the symptom is a MRI-monitored multiple sclerosis disease activity, relapse rate, accumulation of physical disability, frequency of relapses, decreased time to confirmed disease progression, decreased time to confirmed relapse, frequency of clinical exacerbation, brain atrophy, neuronal dysfunction, neuronal injury, neuronal degeneration, neuronal apoptosis, risk for confirmed progression, deterioration of visual function, fatigue, impaired mobility, cognitive impairment, reduction of brain volume, abnormalities observed in whole Brain MTR histogram, deterioration in general health status, functional status, quality of life, and/or symptom severity on work.

In one embodiment, the amount of laquinimod and the amount of flupirtine when taken together is effective to decrease or inhibit reduction of brain volume. In another embodiment, brain volume is measured by percent brain volume change (PBVC).

In one embodiment, the amount of laquinimod and the amount of flupirtine when taken together is effective to increase time to confirmed disease progression. In another embodiment, the time to confirmed disease progression is increased by 20-60%. In yet another embodiment, the time to confirmed disease progression is increased by at least 50%.

In one embodiment, the amount of laquinimod and the amount of flupirtine when taken together is effective to decrease abnormalities observed in whole Brain MTR histogram.

In one embodiment, the accumulation of physical disability is measured by Kurtzke Expanded Disability Status Scale (EDSS) score.

In another embodiment, the accumulation of physical disability is assessed by the time to confirmed disease progression as measured by Kurtzke Expanded Disability Status Scale (EDSS) score. In another embodiment, the subject had an EDSS score of 0-5.5 at baseline. In another embodiment, the subject had an EDSS score of 1.5-4.5 at baseline. In another embodiment, the subject had an EDSS score of 5.5 or greater at baseline. In another embodiment, confirmed disease progression is a 1 point increase of the EDSS score. In yet another embodiment, confirmed disease progression is a 0.5 point increase of the EDSS score.

In one embodiment, impaired mobility is assessed by the Timed-25 Foot Walk test. In another embodiment, impaired mobility is assessed by the 12-Item Multiple Sclerosis Walking Scale (MSWS-12) self-report questionnaire. In another embodiment, impaired mobility is assessed by the Ambulation Index (AI). In another embodiment, impaired mobility is assessed by the Six-Minute Walk (6 MW) Test. In another embodiment, impaired mobility is assessed by the Lower Extremity Manual Muscle Test (LEMMT) Test.

In one embodiment, the amount of laquinimod and the amount of flupirtine when taken together is effective to reduce cognitive impairment. In another embodiment, cognitive impairment is assessed by the Symbol Digit Modalities Test (SDMT) score.

In one embodiment, general health status is assessed by the EuroQoL (EQ5D) questionnaire, Subject Global Impression (SGI) or Clinician Global Impression of Change (CGIC). In another embodiment, functional status is measured by the subject's Short-Form General Health survey (SF-36) Subject Reported Questionnaire score. In another embodiment, quality of life is assessed by SF-36, EQ5D, Subject Global Impression (SGI) or Clinician Global Impression of Change (CGIC). In another embodiment, the subject's SF-36 mental component summary score (MSC) is improved. In another embodiment, the subject's SF-36 physical component summary sore (PSC) is improved. In another embodiment, fatigue is assessed by the EQ5D, the subject's Modified Fatigue Impact Scale (MFIS) score or the French valid versions of the Fatigue Impact Scale (EMIF-SEP) score. In another embodiment, symptom severity on work is measured by the work productivity and activities impairment General Health (WPAI-GH) questionnaire.

In an embodiment, laquinimod is laquinimod sodium. In another embodiment, flupirtine is flupirtine maleate.

In one embodiment, the laquinimod and/or the flupirtine is administered via oral administration. In another embodiment, flupirtine is administered in a slow release form. In another embodiment, flupirtine is administered in an immediate release form. In another embodiment, the laquinimod and/or the flupirtine is administered by injection.

In one particular embodiment of the present invention, if flupirtine is present in the form of a controlled release formulation, the pharmaceutical composition is preferably taken once to 2 times a day. In another embodiment, if flupirtine is present in the form of an immediate release formulation the pharmaceutical composition is preferably taken 3 to 4 times a day.

In an embodiment, the laquinimod and/or the flupirtine is administered periodically. In an embodiment, the laquinimod and/or the flupirtine is administered daily. In another embodiment, the laquinimod and/or the flupirtine is administered more often than once daily. In another embodiment, the laquinimod and/or the flupirtine is administered less often than once daily. In yet another embodiment, the frequency of periodic administration is twice daily, three times daily, four times daily, weekly, twice weekly, three times weekly, etc.

In one embodiment, the ratio by weight of the daily dose of flupirtine to laquinimod is in the range 142:1 to 2000:1. In another embodiment, the amount laquinimod administered is less than 0.6 mg/day. In another embodiment, the amount laquinimod administered is 0.1-40.0 mg/day. In another embodiment, the amount laquinimod administered is 0.1-2.5 mg/day. In another embodiment, the amount laquinimod administered is 0.25-2.0 mg/day. In another embodiment, the amount laquinimod administered is 0.5-1.2 mg/day. In another embodiment, the amount laquinimod administered is 0.25 mg/day. In another embodiment, the amount laquinimod administered is 0.3 mg/day. In another embodiment, the amount laquinimod administered is 0.5 mg/day. In another embodiment, the amount laquinimod administered is 0.6 mg/day. In another embodiment, the amount laquinimod administered is 1.0 mg/day. In another embodiment, the amount laquinimod administered is 1.2 mg/day. In another embodiment, the amount laquinimod administered is 1.5 mg/day. In yet another embodiment, the amount laquinimod administered is 2.0 mg/day.

In one embodiment, the amount flupirtine administered is 10-1000 mg/day. In another embodiment, the amount flupirtine administered is 50-500 mg/day. In another embodiment, the amount flupirtine administered is 100-400 mg/day. In another embodiment, the amount flupirtine administered is 400 mg/day.

In one embodiment, the amount flupirtine administered is a suboptimal dose. In another embodiment, the amount flupirtine administered is less than 1000 mg/day. In another embodiment, the amount flupirtine administered is less than 500 mg/day. In another embodiment, the amount flupirtine administered is less than 400 mg/day. In another embodiment, the amount flupirtine administered is less than 100 mg/day. In another embodiment, the amount flupirtine administered is less than 50 mg/day. In another embodiment, the amount flupirtine administered is less than 10 mg/day.

In an embodiment of the present invention, a loading dose of an amount different form the intended dose is administered for a period of time at the start of the periodic administration. In another embodiment, the loading dose is double the amount of the intended dose.

In an embodiment of the present invention, the subject is receiving laquinimod therapy prior to initiating flupirtine therapy. In another embodiment, the administration of laquinimod substantially precedes the administration of flupirtine. In another embodiment, the subject is receiving flupirtine therapy prior to initiating laquinimod therapy. In another embodiment, the administration of flupirtine substantially precedes the administration of laquinimod. In another embodiment, the subject is receiving flupirtine therapy for at least 8 weeks prior to initiating laquinimod therapy. In another embodiment, the subject is receiving flupirtine therapy for at least 10 weeks prior to initiating laquinimod therapy. In another embodiment, the subject is receiving flupirtine therapy for at least 24 weeks prior to initiating laquinimod therapy. In another embodiment, the subject is receiving flupirtine therapy for at least 28 weeks prior to initiating laquinimod therapy. In another embodiment, the subject is receiving flupirtine therapy for at least 48 weeks prior to initiating laquinimod therapy. In yet another embodiment, the subject is receiving flupirtine therapy for at least 52 weeks prior to initiating laquinimod therapy.

In one embodiment, the method further comprises administration of nonsteroidal anti-inflammatory drugs (NSAIDs), salicylates, slow-acting drugs, gold compounds, hydroxychloroquine, sulfasalazine, combinations of slow-acting drugs, corticosteroids, cytotoxic drugs, immunosuppressive drugs and/or antibodies.

In an embodiment, the periodic administration of laquinimod and flupirtine continues for at least 3 days. In another embodiment, the periodic administration of laquinimod and flupirtine continues for more than 30 days. In another embodiment, the periodic administration of laquinimod and flupirtine continues for more than 42 days. In another embodiment, the periodic administration of laquinimod and flupirtine continues for 8 weeks or more. In another embodiment, the periodic administration of laquinimod and flupirtine continues for at least 12 weeks. In another embodiment, the periodic administration of laquinimod and flupirtine continues for at least 24 weeks. In another embodiment, the periodic administration of laquinimod and flupirtine continues for more than 24 weeks. In another embodiment, the periodic administration of laquinimod and flupirtine continues for 6 months or more.

In one embodiment, the administration of laquinimod and flupirtine inhibits a symptom of relapsing multiple sclerosis by at least 20%. In another embodiment, the administration of laquinimod and flupirtine inhibits a symptom of relapsing multiple sclerosis by at least 30%. In another embodiment, the administration of laquinimod and flupirtine inhibits a symptom of relapsing multiple sclerosis by at least 50%. In another embodiment, the administration of laquinimod and flupirtine inhibits a symptom of relapsing multiple sclerosis by at least 70%. In another embodiment, the administration of laquinimod and flupirtine inhibits a symptom of relapsing multiple sclerosis by more than 100%. In another embodiment, the administration of laquinimod and flupirtine inhibits a symptom of relapsing multiple sclerosis by more than 300%. In another embodiment, the administration of laquinimod and flupirtine inhibits a symptom of relapsing multiple sclerosis by more than 1000%.

In one embodiment, each of the amount of laquinimod or pharmaceutically acceptable salt thereof when taken alone, and the amount of flupirtine when taken alone is effective to treat the subject.

In another embodiment, either the amount of laquinimod or pharmaceutically acceptable salt thereof when taken alone, the amount of flupirtine when taken alone, or each such amount when taken alone is not effective to treat the subject.

In another embodiment, the subject is a human patient.

The subject invention also provides a package comprising: a) a first pharmaceutical composition comprising an amount of laquinimod and a pharmaceutically acceptable carrier; b) a second pharmaceutical composition comprising an amount of flupirtine and a pharmaceutically acceptable carrier; and c) instructions for use of the first and second pharmaceutical compositions together to treat a subject afflicted with multiple sclerosis or presenting a clinically isolated syndrome.

In an embodiment of the present invention, the first pharmaceutical composition, the second pharmaceutical composition, or both the first and the second pharmaceutical composition are in an aerosol, an inhalable powder, an injectable, a liquid, a solid, a capsule or a tablet form. In another embodiment, the first pharmaceutical composition, the second pharmaceutical composition, or both the first and the second pharmaceutical composition are in a liquid or a solid form. In another embodiment, the first pharmaceutical composition, the second pharmaceutical composition, or both the first and the second pharmaceutical composition are in capsule form or in tablet form.

In an embodiment, the tablets are coated with a coating which inhibits oxygen from contacting the core. In another embodiment, the coating comprises a cellulosic polymer, a detackifier, a gloss enhancer, or pigment.

In one embodiment, the first pharmaceutical composition further comprises mannitol. In another embodiment, the first pharmaceutical composition further comprises an alkalinizing agent. In another embodiment, the alkalinizing agent is meglumine. In another embodiment, the first pharmaceutical composition further comprises an oxidation reducing agent.

In one embodiment, the first pharmaceutical composition is stable and free of an alkalinizing agent or an oxidation reducing agent. In another embodiment, the first pharmaceutical composition is free of an alkalinizing agent and free of an oxidation reducing agent.

In one embodiment, the first pharmaceutical composition is stable and free of disintegrant. In another embodiment, the first pharmaceutical composition further comprises a lubricant. In another embodiment, the lubricant is present in the composition as solid particles. In another embodiment, the lubricant is sodium stearyl fumarate or magnesium stearate.

In one embodiment, the first pharmaceutical composition further comprises a filler. In another embodiment, the filler is present in the composition as solid particles. In another embodiment, the filler is lactose, lactose monohydrate, starch, isomalt, mannitol, sodium starch glycolate, sorbitol, lactose spray dried, lactose anhydrouse, or a combination thereof. In another embodiment, the filler is mannitol or lactose monohydrate.

In an embodiment, the package further comprises a desiccant. In another embodiment, the desiccant is silica gel.

In an embodiment, the first pharmaceutical composition is stable and has a moisture content of no more than 4%. In another embodiment, laquinimod is present in the composition as solid particles. In another embodiment, the package is a sealed packaging having a moisture permeability of not more than 15 mg/day per liter.

In an embodiment, the sealed package is a blister pack in which the maximum moisture permeability is no more than 0.005 mg/day. In another embodiment, the sealed package is a bottle. In another embodiment, the bottle is closed with a heat induction liner. In another embodiment, the sealed package comprises an HDPE bottle. In another embodiment, the sealed package comprises an oxygen absorbing agent. In another embodiment, the oxygen absorbing agent is iron.

In one embodiment, the amount of laquinimod in the first composition is less than 0.6 mg. In another embodiment, the amount of laquinimod in the first composition is 0.1-40.0 mg. In another embodiment, the amount of laquinimod in the first composition is 0.1-2.5 mg. In another embodiment, the amount of laquinimod in the first composition is 0.25-2.0 mg. In another embodiment, the amount of laquinimod in the first composition is 0.5-1.2 mg. In another embodiment, the amount of laquinimod in the first composition is 0.25 mg. In another embodiment, the amount of laquinimod in the first composition is 0.3 mg. In another embodiment, the amount of laquinimod in the first composition is 0.5 mg. In another embodiment, the amount of laquinimod in the first composition is 0.6 mg. In another embodiment, the amount of laquinimod in the first composition is 1.0 mg. In another embodiment, the amount of laquinimod in the first composition is 1.2 mg. In another embodiment, the amount of laquinimod in the first composition is 1.5 mg. In another embodiment, the amount of laquinimod in the first composition is 2.0 mg.

In one embodiment, the amount of flupirtine is 10-1000 mg. In another embodiment, the amount of flupirtine is 50-500 mg. In another embodiment, the amount of flupirtine is 100-400 mg. In another embodiment, the amount of flupirtine is 400 mg.

In some embodiments, the amount of flupirtine in the pharmaceutical composition is from 10 mg to 1000 mg, such as from 50 mg to 500 mg. In one embodiment, the amount of flupirtine in the pharmaceutical composition is preferably from 100 mg to 400 mg. In an embodiment, if flupirtine is present in the form of a controlled release formulation, the most preferred amount of flupirtine in the pharmaceutical composition is 400 mg. In another embodiment, if flupirtine is present in the form of a immediate release formulation, the most preferred amount of flupirtine in the pharmaceutical composition is 100 mg.

In one embodiment of the present invention, the amount of laquinimod and the amount of flupirtine are prepared to be administered simultaneously, contemporaneously or concomitantly.

The subject invention also provides laquinimod for use as an add-on therapy or in combination with flupirtine in treating a subject afflicted with multiple sclerosis or presenting a clinically isolated syndrome, and flupirtine for use as an add-on therapy or in combination with laquinimod in treating a subject afflicted with multiple sclerosis or presenting a clinically isolated syndrome.

The subject invention also provides a pharmaceutical composition comprising an amount of laquinimod and an amount of flupirtine for use in treating a subject afflicted with multiple sclerosis or presenting a clinically isolated syndrome, wherein the laquinimod and the flupirtine are prepared to be administered simultaneously, contemporaneously or concomitantly.

The subject invention also provides a pharmaceutical composition comprising an amount of laquinimod and an amount of flupirtine.

In one embodiment, the ratio of flupirtine to laquinimod by weight is in the range 142:1 to 2000:1. In another embodiment, laquinimod is laquinimod sodium. In another embodiment, flupirtine is flupirtine maleate.

In an embodiment, the pharmaceutical composition is in an aerosol, an inhalable powder, an injectable, a liquid, a solid, a capsule or a tablet form. In another embodiment, the pharmaceutical composition is in a liquid or a solid form. In another embodiment, it is in capsule form or in tablet form.

In one embodiment, the tablets are coated with a coating which inhibits oxygen from contacting the core. In another embodiment, the coating comprises a cellulosic polymer, a detackifier, a gloss enhancer, or pigment.

In an embodiment, the pharmaceutical composition further comprises mannitol. In another embodiment, the pharmaceutical composition further comprises an alkalinizing agent. In another embodiment, the alkalinizing agent is meglumine. In another embodiment, the pharmaceutical composition further comprises an oxidation reducing agent.

In one embodiment, the pharmaceutical composition is free of an alkalinizing agent or an oxidation reducing agent. In another embodiment, the pharmaceutical composition is free of an alkalinizing agent and free of an oxidation reducing agent.

In one embodiment, the pharmaceutical composition is stable and free of disintegrant. In another embodiment, the pharmaceutical composition further comprises a lubricant. In another embodiment, the lubricant is present in the composition as solid particles. In another embodiment, the lubricant is sodium stearyl fumarate or magnesium stearate.

In one embodiment, the pharmaceutical composition further comprises a filler. In another embodiment, the filler is present in the composition as solid particles. In another embodiment, the filler is lactose, lactose monohydrate, starch, isomalt, mannitol, sodium starch glycolate, sorbitol, lactose spray dried, lactose anhydrouse, or a combination thereof. In another embodiment, the filler is mannitol or lactose monohydrate.

In one embodiment, the amount of laquinimod in the composition is less than 0.6 mg. In another embodiment, the amount of laquinimod in the composition is 0.1-40.0 mg. In another embodiment, the amount of laquinimod in the composition is 0.1-2.5 mg. In another embodiment, the amount of laquinimod in the composition is 0.25-2.0 mg. In another embodiment, the amount of laquinimod in the composition is 0.5-1.2 mg. In another embodiment, the amount of laquinimod in the composition is 0.25 mg. In another embodiment, the amount of laquinimod in the composition is 0.3 mg. In another embodiment, the amount of laquinimod in the composition is 0.5 mg. In another embodiment, the amount of laquinimod in the composition is 0.6 mg. In another embodiment, the amount of laquinimod in the composition is 1.0 mg. In another embodiment, the amount of laquinimod in the composition is 1.2 mg. In another embodiment, the amount of laquinimod in the composition is 1.5 mg. In another embodiment, the amount of laquinimod in the composition is 2.0 mg.

In an embodiment, the amount of flupirtine in the composition is 10-1000 mg. In another embodiment, the amount of flupirtine in the composition is 50-500 mg. In another embodiment, the amount of flupirtine in the composition is 100-400 mg. In yet another embodiment, the amount of flupirtine in the composition is 400 mg.

The subject invention also provides use of an amount of laquinimod and an amount of flupirtine in the preparation of a combination for treating a subject afflicted with multiple sclerosis or presenting a clinically isolated syndrome wherein the laquinimod and the flupirtine are prepared to be administered simultaneously, contemporaneously or concomitantly.

The subject invention also provides a pharmaceutical composition comprising an amount of laquinimod for use in treating a subject afflicted with multiple sclerosis or presenting a clinically isolated syndrome as an add-on therapy or in combination with flupirtine.

The subject invention also provides a pharmaceutical composition comprising an amount of flupirtine for use treating a subject afflicted with multiple sclerosis or presenting a clinically isolated syndrome as an add-on therapy or in combination with laquinimod.

The subject invention also provides a pharmaceutical composition comprising an amount of laquinimod for use in treating a subject afflicted with multiple sclerosis or presenting a clinically isolated syndrome simultaneously, contemporaneously or concomitantly with flupirtine.

The subject invention also provides a pharmaceutical composition comprising an amount of flupirtine for use treating a subject afflicted with multiple sclerosis or presenting a clinically isolated syndrome simultaneously, contemporaneously or concomitantly with laquinimod.

The subject invention also provides a therapeutic package for dispensing to, or for use in dispensing to, a subject afflicted with multiple sclerosis or presenting a clinically isolated syndrome, which comprises: a) one or more unit doses, each such unit dose comprising: i) an amount of laquinimod and ii) an amount of flupirtine, wherein the respective amounts of said laquinimod and said flupirtine in said unit dose are effective, upon concomitant administration to said subject, to treat the subject, and b) a finished pharmaceutical container therefor, said container containing said unit dose or unit doses, said container further containing or comprising labeling directing the use of said package in the treatment of said subject.

In one embodiment, the respective amounts of said laquinimod and said flupirtine in said unit dose when taken together is more effective to treat the subject than when compared to the administration of said laquinimod in the absence of said flupirtine or the administration of said flupirtine in the absence of said laquinimod.

The subject invention also provides a pharmaceutical composition in unit dosage form, useful in treating a subject afflicted with multiple sclerosis or presenting a clinically isolated syndrome, which comprises: a) an amount of laquinimod; b) an amount of flupirtine, wherein the respective amounts of said laquinimod and said flupirtine in said composition are effective, upon concomitant administration to said subject of one or more of said unit dosage forms of said composition, to treat the subject.

In one embodiment, the respective amounts of said laquinimod and said flupirtine in said unit dose when taken together is more effective to treat the subject than when compared to the administration of said laquinimod in the absence of said flupirtine or the administration of said flupirtine in the absence of said laquinimod.

Flupirtine can be administered by way of oral, sublingual, injection including subcutaneous, intramuscular and intravenous, topical, intratracheal, intranasal, transdermal or rectal administration. Flupirtine may be administered in admixture with conventional pharmaceutical carriers. The appropriate unit forms of administration include forms for oral administration, such as tablets, gelatin capsules, powders, granules and solutions or suspensions to be taken orally, forms for sublingual, buccal, intratracheal or intranasal administration, forms for injection including subcutaneous, intramuscular or intravenous administration and forms for rectal administration. For topical application, flupirtine can be used in creams, ointments or lotions. In one particular embodiment, oral administration is preferred.

Examples of suitable acids for preparing physiologically tolerated flupirtine salts include hydrohalic acids, sulphuric acid, phosphoric acids, nitric acid, perchloric acid, organic mono-, di- or tricarboxylic acids of the aliphatic, alicyclic, aromatic or heterocyclic series, and sulphonic acids. In a particular embodiment, preferred examples of suitable acids are formic, acetic, propionic, succinic, glycolic, lactic, malic, tartaric, citric, ascorbic, maleic, fumaric, hydroxymaleic, pyruvic, phenylacetic, benzoic, p-aminosalicylic, embonic, methanesulphonic, ethanesulphonic, hydroxyethanesulphonic, ethylenesulphonic, halobenzenesulphonic, toluenesulphonic, naphthalenesulphonic acids, sulphanilic acid and hydrochloric acid.

In an embodiment of the invention, the physiologically tolerated flupirtine salt is the formate, acetate, propionate, succinate, glycolate, lactate, malate, tartrate, citrate, maleate, mesylate, besilate, phosphate, fumarate, pyruvate, phenylacetate, benzoate, embonate, methanesulphonate, ethanesulphonate, hydroxyethanesulphonate, ethylenesulphonate, halobenzonesulphonate, toluenesulphonate, naphthalenesulphonate, aminobenzenesulphonate or chloride of flupirtine. The production of flupirtine and its physiologically usable salts are described e.g., in DE 17 95 858 C2, DE 31 33 519 C2, DE 34 16 609 A1 and PCT International Application Publication No. WO 2011/157719, which are hereby incorporated by reference into this application.

Laquinimod mixtures, compositions, and the process for the manufacture thereof are described in, e.g., U.S. Pat. No. 6,077,851, U.S. Pat. No. 7,884,208, U.S. Pat. No. 7,989,473, U.S. Pat. No. 8,178,127, U.S. Application Publication No. 2010-0055072, U.S. Application Publication No. 2012-0010238, and U.S. Application Publication No. 2012-0010239, each of which is hereby incorporated by reference in its entireties into this application.

Use of laquinimod for treatment of various conditions, and the corresponding dosages and regimens, are described in U.S. Pat. No. 6,077,851 (multiple sclerosis, insulin-dependent diabetes mellitus, systemic lupus erythematosus, rheumatoid arthritis, inflammatory bowel disease, psoriasis, inflammatory respiratory disorder, atherosclerosis, stroke, and Alzheimer's disease), U.S. Application Publication No. 2011-0027219 (Crohn's disease), U.S. Application Publication No. 2010-0322900 (Relapsing-remitting multiple sclerosis), U.S. Application Publication No. 2011-0034508 (brain-derived neurotrophic factor (BDNF)-related diseases), U.S. Application Publication No. 2011-0218179 (active lupus nephritis), U.S. Application Publication No. 2011-0218203 (rheumatoid arthritis), U.S. Application Publication No. 2011-0217295 (active lupus arthritis), and U.S. Application Publication No. 2012-0142730 (reducing fatigue, improving quality of life, and providing neuroprotection in MS patients), each of which is hereby incorporated by reference in its entireties into this application.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Pat. No. 7,589,208 and PCT International Application Publication No. WO 2005/074899, which are hereby incorporated by reference into this application.

Laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit can be in a form suitable for oral administration. Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar. Capsule or tablets can be easily formulated and can be made easy to swallow or chew; other solid forms include granules, and bulk powders.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Pat. No. 7,589,208, PCT International Application Publication Nos. WO 2005/074899, WO 2007/047863, and 2007/146248.

General techniques and compositions for making dosage forms useful in the present invention are described in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds).; Modern Pharmaceutics Drugs and the Pharmaceutical Sciences, Vol. 40 (Gilbert S. Banker, Christopher T. Rhodes, Eds). These references in their entireties are hereby incorporated by reference into this application.

Disclosed is a method for treating a subject, e.g., human patient, afflicted with multiple sclerosis, e.g., relapsing multiple sclerosis or presenting a CIS using laquinimod with flupirtine which provides a more efficacious treatment than each agent alone. The use of laquinimod for multiple sclerosis had been previously suggested in, e.g., U.S. Pat. No. 6,077,851. However, the inventors have surprisingly found that the combination of laquinimod and flupirtine is particularly effective for the treatment of a subject afflicted with MS or presenting a CIS as compared to each agent alone.

### Terms

As used herein, and unless stated otherwise, each of the following terms shall have the definition set forth below.

As used herein, “laquinimod” means laquinimod acid or a pharmaceutically acceptable salt thereof.

As used herein, “flupirtine” means flupirtine base or a pharmaceutically acceptable salt thereof.

As used herein, an “amount” or “dose” of laquinimod or flupirtine as measured in milligrams refers to the milligrams of laquinimod acid or flupirtine base present in a preparation, regardless of the form of the preparation. A “dose of 0.6 mg laquinimod” means the amount of laquinimod acid in a preparation is 0.6 mg, regardless of the form of the preparation. Thus, when in the form of a salt, e.g. a laquinimod sodium salt, the weight of the salt form necessary to provide a dose of 0.6 mg laquinimod would be greater than 0.6 mg (e.g., 0.64 mg) due to the presence of the additional salt ion. Similarly, when in the form of a salt, e.g. flupirtine maleate, the weight of the salt form necessary to provide a dose of 400 mg flupirtine would be greater than 400 mg (e.g., 430 mg) due to the presence of the additional salt ion.

As used herein, a “unit dose”, “unit doses” and “unit dosage form(s)” mean a single drug administration entity/entities.

As used herein, “about” in the context of a numerical value or range means±10% of the numerical value or range recited or claimed.

As used herein, a composition that is “free” of a chemical entity means that the composition contains, if at all, an amount of the chemical entity which cannot be avoided although the chemical entity is not part of the formulation and was not affirmatively added during any part of the manufacturing process. For example, a composition which is “free” of an alkalizing agent means that the alkalizing agent, if present at all, is a minority component of the composition by weight. Preferably, when a composition is “free” of a component, the composition comprises less than 0.1 wt %, 0.05 wt %, 0.02 wt %, or 0.01 wt % of the component.

As used herein, “alkalizing agent” is used interchangeably with the term “alkaline-reacting component” or “alkaline agent” and refers to any pharmaceutically acceptable excipient which neutralizes protons in, and raises the pH of, the pharmaceutical composition in which it is used.

As used herein, “oxidation reducing agent” refers to a group of chemicals which includes an “antioxidant”, a “reduction agent” and a “chelating agent”.

As used herein, “antioxidant” refers to a compound or molecule that inhibits the oxidation of other molecules. Examples of antoxidants include tocopherol, methionine, glutathione, tocotrienol, dimethyl glycine, betaine, butylated hydroxyanisole, butylated hydroxytoluene, turmerin, vitamin E, ascorbyl palmitate, tocopherol, deteroxime mesylate, methyl paraben, ethyl paraben, butylated hydroxyanisole, butylated hydroxytoluene, propyl gallate, sodium or potassium metabisulfite, sodium or potassium sulfite, alpha tocopherol or derivatives thereof, sodium ascorbate, disodium edentate, BHA (butylated hydroxyanisole), a pharmaceutically acceptable salt or ester of the mentioned compounds, and mixtures thereof.

The term “antioxidant” as used herein is also exemplified by flavonoids such as those selected from the group of quercetin, morin, naringenin and hesperetin, taxifolin, afzelin, quercitrin, myricitrin, genistein, apigenin and biochanin A, flavone, flavopiridol, isoflavonoids such as the soy isoflavonoid, genistein, catechins such as the tea catechin epigallocatechin gallate, flavonol, epicatechin, hesperetin, chrysin, diosmin, hesperidin, luteolin, and rutin.

As used herein, “reduction agent” refers to a compound exemplified by the group consisting of thiol-containing compound, thioglycerol, mercaptoethanol, thioglycol, thiodiglycol, cysteine, thioglucose, dithiothreitol (DTT), dithio-bis-maleimidoethane (DTME), 2,6-di-tert-butyl-4-methylphenol (BHT), sodium dithionite, sodium bisulphite, formamidine sodium metabisulphite, and ammonium bisulphite.”

As used herein, “chelating agent” refers to a compound exemplified by the group consisting of penicillamine, trientine, N,N′-diethyldithiocarbamate (DDC), 2,3,2′-tetraamine (2,3,2′-tet), neocuproine, N,N,N′,N′-tetrakis(2-pyridylmethyl)ethylenediamine (TPEN), 1,10-phenanthroline (PHE), tetraethylenepentamine, triethylenetetraamine and tris(2-carboxyethyl) phosphine (TCEP), ferrioxamine, CP94, EDTA, deferoxainine B (DFO) as the methanesulfonate salt (also known as desferrioxanilne B mesylate (DFOM)), desferal from Novartis (previously Ciba-Giegy), and apoferritin.

As used herein, a pharmaceutical composition is “stable” when the composition preserves the physical stability/integrity and/or chemical stability/integrity of the active pharmaceutical ingredient during storage. Furthermore, “stable pharmaceutical composition” is characterized by its level of degradation products not exceeding 5% at 40° C./75% RH after 6 months or 3% at 55° C./75% RH after two weeks, compared to their level in time zero.

As used herein, “combination” means an assemblage of reagents for use in therapy either by simultaneous or contemporaneous administration. Simultaneous administration refers to administration of an admixture (whether a true mixture, a suspension, an emulsion or other physical combination) of the laquinimod and the flupirtine. In this case, the combination may be the admixture or separate containers of the laquinimod and the flupirtine that are combined just prior to administration. Contemporaneous administration refers to the separate administration of the laquinimod and the flupirtine at the same time, or at times sufficiently close together that a synergistic activity relative to the activity of either the laquinimod or the flupirtine alone is observed.

As used herein, “concomitant administration” or administering “concomitantly” means the administration of two agents given in close enough temporal proximately to allow the individual therapeutic effects of each agent to overlap.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time. For example, adding laquinimod therapy to a patient already receiving flupirtine therapy.

As used herein, “effective” when referring to an amount of laquinimod and/or flupirtine refers to the quantity of laquinimod and/or flupirtine that is sufficient to yield a desired therapeutic response. Efficacy can be measured by an improvement of a symptom of multiple sclerosis. Such symptoms can include a MRI-monitored multiple sclerosis disease activity, relapse rate, accumulation of physical disability, frequency of relapses, time to confirmed disease progression, time to confirmed relapse, frequency of clinical exacerbation, brain atrophy, neuronal dysfunction, neuronal injury, neuronal degeneration, neuronal apoptosis, risk for confirmed progression, visual function, fatigue, impaired mobility, cognitive impairment, brain volume, abnormalities observed in whole Brain MTR histogram, general health status, functional status, quality of life, and/or symptom severity on work.

In an embodiment, an effective amount is an amount that is sufficient to decrease or inhibit reduction of brain volume (optionally brain volume is measured by percent brain volume change (PBVC)), increase time to confirmed disease progression (e.g., by 20-60% or at least 50%), decrease abnormalities observed in whole Brain MTR histogram, decrease the accumulation of physical disability (optionally measured by Kurtzke Expanded Disability Status Scale (EDSS) score, e.g., wherein the accumulation of physical disability is assessed by the time to confirmed disease progression as measured by Kurtzke Expanded Disability Status Scale (EDSS) score), improve impaired mobility (optionally assessed by the Timed-25 Foot Walk test, the 12-Item Multiple Sclerosis Walking Scale (MSWS-12) self-report questionnaire, the Ambulation Index (AI), the Six-Minute Walk (6 MW) Test, or the Lower Extremity Manual Muscle Test (LEMMT) Test), reduce cognitive impairment (optionally assessed by the Symbol Digit Modalities Test (SDMT) score), improve general health (optionally assessed by the EuroQoL (EQ5D) questionnaire, Subject Global Impression (SGI) or Clinician Global Impression of Change (CGIC)), improve functional status (optionally measured by the subject's Short-Form General Health survey (SF-36) Subject Reported Questionnaire score), improve quality of life (optionally assessed by SF-36, EQ5D, Subject Global Impression (SGI) or Clinician Global Impression of Change (CGIC)), improve the subject's SF-36 mental component summary score (MSC) and/or SF-36 physical component summary sore (PSC), reduce level of fatigue (optionally assessed by the EQ5D, the subject's Modified Fatigue Impact Scale (MFIS) score or the French valid versions of the Fatigue Impact Scale (EMIF-SEP) score), or improve symptom severity on work (optionally measured by the work productivity and activities impairment General Health (WPAI-GH) questionnaire).

“Administering to the subject” or “administering to the (human) patient” means the giving of, dispensing of, or application of medicines, drugs, or remedies to a subject/patient to relieve, cure, or reduce the symptoms associated with a condition, e.g., a pathological condition. The administration can be periodic administration. As used herein, “periodic administration” means repeated/recurrent administration separated by a period of time. The period of time between administrations is preferably consistent from time to time. Periodic administration can include administration, e.g., once daily, twice daily, three times daily, four times daily, weekly, twice weekly, three times weekly, four times a week and so on, etc.

“Treating” as used herein encompasses, e.g., inducing inhibition, regression, or stasis of a disease or disorder, e.g., Relapsing MS (RMS), or alleviating, lessening, suppressing, inhibiting, reducing the severity of, eliminating or substantially eliminating, or ameliorating a symptom of the disease or disorder. “Treating” as applied to patients presenting CIS can mean delaying the onset of clinically definite multiple sclerosis (CDMS), delaying the progression to CDMS, reducing the risk of conversion to CDMS, or reducing the frequency of relapse in a patient who experienced a first clinical episode consistent with multiple sclerosis and who has a high risk of developing CDMS.

“Inhibition” of disease progression or disease complication in a subject means preventing or reducing the disease progression and/or disease complication in the subject.

A “symptom” associated with MS or RMS includes any clinical or laboratory manifestation associated with MS or RMS and is not limited to what the subject can feel or observe.

As used herein, “a subject afflicted with multiple sclerosis” or “a subject afflicted with relapsing multiple sclerosis” means a subject who has been clinically diagnosed to have multiple sclerosis or relapsing multiple sclerosis (RMS), which includes relapsing-remitting multiple sclerosis (RRMS) and Secondary Progressive multiple sclerosis (SPMS).

As used herein, a subject at “baseline” is as subject prior to administration of laquinimod.

A “patient at risk of developing MS” (i.e. clinically definite MS) as used herein is a patient presenting any of the known risk factors for MS. The known risk factors for MS include any one of a clinically isolated syndrome (CIS), a single attack suggestive of MS without a lesion, the presence of a lesion (in any of the CNS, PNS, or myelin sheath) without a clinical attack, environmental factors (geographical location, climate, diet, toxins, sunlight), genetics (variation of genes encoding HLA-DRB1, IL7R-alpha and IL2R-alpha), and immunological components (viral infection such as by Epstein-Barr virus, high avidity CD4 T cells, CD8* T cells, anti-NF-L, anti-CSF 114(Glc)).

“Clinically isolated syndrome (CIS)” as used herein refers to 1) a single clinical attack (used interchangeably herein with “first clinical event” and “first demyelinating event”) suggestive of MS, which, for example, presents as an episode of optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning, and 2) at least one lesion suggestive of MS. In a specific example, CIS diagnosis would be based on a single clinical attack and at least 2 lesions suggestive of MS measuring 6 mm or more in diameter.

“Relapse Rate” is the number of confirmed relapses per unit time. “Annualized relapse rate” is the mean value of the number of confirmed relapses of each patient multiplied by 365 and divided by the number of days that patient is on the study drug.

“Expanded Disability Status Scale” or “EDSS” is a rating system that is frequently used for classifying and standardizing the condition of people with multiple sclerosis. The score ranges from 0.0 representing a normal neurological exam to 10.0 representing death due to MS. The score is based upon neurological testing and examination of functional systems (FS), which are areas of the central nervous system which control bodily functions. The functional systems are: Pyramidal (ability to walk), Cerebellar (coordination), Brain stem (speech and swallowing), Sensory (touch and pain), Bowel and bladder functions, Visual, Mental, and Other (includes any other neurological findings due to MS) (Kurtzke J F, 1983).

A “confirmed progression” of EDSS, or “confirmed disease progression” as measured by EDSS score is defined as a 1 point increase from baseline EDSS if baseline EDSS was between 0 and 5.0, or a 0.5 point increase if baseline EDSS was 5.5. In order to be considered a confirmed progression, the change (either 1 point or 0.5 points) must be sustained for at least 3 months. In addition, confirmation of progression cannot be made during a relapse.

“Adverse event” or “AE” means any untoward medical occurrence in a clinical trial subject administered a medicinal product and which does not have a causal relationship with the treatment. An adverse event can therefore be any unfavorable and unintended sign including an abnormal laboratory finding, symptom, or diseases temporally associated with the use of an investigational medicinal product, whether or not considered related to the investigational medicinal product.

“Gd-enhancing lesion” refers to lesions that result from a breakdown of the blood-brain barrier, which appear in contrast studies using gandolinium contrast agents. Gandolinium enhancement provides information as to the age of a lesion, as Gd-enhancing lesions typically occur within a six week period of lesion formation.

“Magnetization Transfer Imaging” or “MTI” is based on the magnetization interaction (through dipolar and/or chemical exchange) between bulk water protons and macromolecular protons. By applying an off resonance radio frequency pulse to the macromolecular protons, the saturation of these protons is then transferred to the bulk water protons. The result is a decrease in signal (the net magnetization of visible protons is reduced), depending on the magnitude of MT between tissue macromolecules and bulk water. “MT” or “Magnetization Transfer” refers to the transfer of longitudinal magnetization from the hydrogen nuclei of water that have restricted motion to the hydrogen nuclei of water that moves with many degrees of freedom. With MTI, the presence or absence of macromolecules (e.g. in membranes or brain tissue) can be seen (Mehta, 1996; Grossman, 1994).

“Magnetization Resonance Spectroscopy” or “MRS” is a specialized technique associated with magnetic resonance imaging (MRI). MRS is used to measure the levels of different metabolites in body tissues. The MR signal produces a spectrum of resonances that correspond to different molecular arrangements of the isotope being “excited”. This signature is used to diagnose certain metabolic disorders, especially those affecting the brain, (Rosen, 2007) as well as to provide information on tumor metabolism (Golder, 2007).

As used herein “mobility” refers to any ability relating to walking, walking speed, gait, strength of leg muscles, leg function and the ability to move with or without assistance. Mobility can be evaluated by one or more of several tests including but not limited to Ambulation Index, Time 25 foot walk, Six-Minute Walk (6 MW), Lower Extremity Manual Muscle Test (LEMMT) and EDSS. Mobility can also be reported by the subject, for example by questionnaires, including but not limited to 12-Item Multiple Sclerosis Walking Scale (MSWS-12). Impaired Mobility refers to any impairment, difficulty or disability relating to mobility.

“T1-weighted MRI image” refers to an MR-image that emphasizes T1 contrast by which lesions may be visualized. Abnormal areas in a T1-weighted MRI image are “hypointense” and appear as dark spots. These spots are generally older lesions.

“T2-weighted MRI image” refers to an MR-image that emphasizes T2 contrast by which lesions may be visualized. T2 lesions represent new inflammatory activity.

The “Six-Minute Walk (6 MW) Test” is a commonly used test developed to assess exercise capacity in patients with COPD (Guyatt, 1985). It has been used also to measure mobility in multiple sclerosis patients (Clinical Trials Website).

The “Timed-25 Foot Walk” or “T25-FW” is a quantitative mobility and leg function performance test based on a timed 25-walk. The patient is directed to one end of a clearly marked 25-foot course and is instructed to walk 25 feet as quickly as possible, but safely. The time is calculated from the initiation of the instruction to start and ends when the patient has reached the 25-foot mark. The task is immediately administered again by having the patient walk back the same distance. Patients may use assistive devices when doing this task. The score for the T25-FW is the average of the two completed trials. This score can be used individually or used as part of the MSFC composite score (National MS Society Website).

One of the central symptoms of multiple sclerosis is fatigue. Fatigue can be measured by several tests including but not limited to decrease of French valid versions of the Fatigue Impact Scale (EMIF-SEP) score, and European Quality of Life (EuroQoL) Questionnaire (EQ5D). Other tests, including but not limited to Clinician Global Impression of Change (CGIC) and Subject Global Impression (SGI), as well as EQ-5D, can be used to evaluate the general health status and quality of life of MS patients.

“Ambulation Index” or “AI” is a rating scale developed by Hauser et al. to assess mobility by evaluating the time and degree of assistance required to walk 25 feet. Scores range from 0 (asymptomatic and fully active) to 10 (bedridden). The patient is asked to walk a marked 25-foot course as quickly and safely as possible. The examiner records the time and type of assistance (e.g., cane, walker, crutches) needed. (Hauser, 1983)

“EQ-5D” is a standardized questionnaire instrument for use as a measure of health outcome applicable to a range of health conditions and treatments. It provides a simple descriptive profile and a single index value for health status that can be used in the clinical and economic evaluation of health care as well as population health surveys. EQ-5D was developed by the “EuroQoL” Group which comprises a network of international, multilingual, multidisciplinary researchers, originally from seven centers in England, Finland, the Netherlands, Norway and Sweden. The EQ-5D questionnaire is in the public domain and can be obtained from EuroQoL.

“SF-36” is a multi-purpose, short-form health survey with 36 questions which yields an 8-scale profile of functional health and well-being scores as well as psychometrically-based physical and mental health summary measures and a preference-based health utility index. It is a generic measure, as opposed to one that targets a specific age, disease, or treatment group. The survey is developed by and can be obtained from QualityMetric, Inc. of Providence, R.I.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

It is understood that where a parameter range is provided, all integers within that range, and tenths thereof, are also provided by the invention. For example, “0.1-2.5 mg/day” includes 0.1 mg/day, 0.2 mg/day, 0.3 mg/day, etc. up to 2.5 mg/day.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Assessment of Efficacy of Laquinimod Alone or In-Combination with Flupirtine in MOG-Induced EAE

In this experiment, MOG-induced EAE Mice were treated with laquinimod alone or with add on flupirtine to assess the efficacy of laquinimod alone or in combination with flupirtine. MOG-induced Experimental Autoimmune Encephalomyelitis (EAE) in the C57B1 strain of mice is an established EAE model to test the efficacy of candidate molecules for MS treatment.

The dosages were chosen based on known effective dose amounts for laquinimod (0.6 mg/day) and for flupirtine (400 mg/day) in humans (U.S. Patent Application Publication 2008-0279952; U.S. Patent Application Publication 2010-0322900). The National Institutes of Health (NIH) provides a table of Equivalent Surface Area Dosage Conversion Factors below (Table 1) which provides conversion factors that account for surface area to weight ratios between species.

TABLE 1
Equivalent Surface Area Dosage Conversion Factors

| | | To | | | | |
|---|---|---|---|---|---|---|
| | | Mouse | | Monkey | | |
| | | 20 g | Rat 150 g | 3 kg | Dog 8 kg | Man 60 kg |
| FROM | Mouse | 1 | ½ | ¼ | ⅙ | 1/12 |
| | Rat | 2 | 1 | ½ | ¼ | 1/7 |
| | Monkey | 4 | 2 | 1 | ⅗ | ⅓ |
| | Dog | 6 | 4 | 1⅔ | 1 | ½ |
| | Man | 12 | 7 | 3 | 2 | 1 |

The data from this mice study is representative of what can be expected in human patients with the treatment of laquinimod and flupirtine at the corresponding human dosages.

### Procedure

Disease was induced in all mice by the injection of the encephalitogenic emulsion (MOG/CFA) and intraperitoneal injection of pertussis toxin on the first day and 48 hours later.
Flupirtine at a dose level of 30 mg/kg was administered as an oral dose, once daily (QD).
Laquinimod at dose levels of 1 and 5 mg/kg were administered by the oral route, once daily (QD).
Both flupirtine and laquinimod were administered prophylactic from disease induction—Day 1 until termination of the study.

### Induction of EAE:

EAE was induced by subcutaneous injection of encephalitogenic emulsion at a volume of 0.2 ml/mouse in the flanks at two injection sites. On the day of induction, pertussis toxin was injected i.p. at a volume dose of 0.2 ml/mouse. The injection of the pertussis toxin was repeated after 48 hours.

### Test Procedure:

Day 0: Subcutaneous injection of MOG into the flanks, i.p. injection of Pertussis toxin, beginning of daily laquinimod treatment.
Day 2: i.p. injection of Pertussis toxin.
Day 8: Initiation of scoring of mice for EAE clinical signs.
Day 30: Termination of study.

### Materials:

1. Flupirtine
2. Laquinimod
3. Mycobacterium tuberculosis (MT), Difco
4. Pertussis toxin, Sigma
5. MOG 35-55, Mnf Novatide
6. Complete Freund's Adjuvant (CFA), Sigma
7. Methocel (methyl cellulose (MC)), Sigma
8. Saline, Mnf-DEMO S.A

### Experimental Animals:

Healthy, nulliparous, non-pregnant female mice of the C57BL/6 strain were used in the study.

The animals weighed 17-20 grams, and were approximately 7 weeks old on receipt.

The body weights of the animals were recorded on the day of delivery.

Overtly healthy animals were assigned to study groups arbitrarily before treatment commenced.

The mice were individually identified by using ear tags. A color-coded card on each cage gave information including cage number, group number and identification.

### EAE Induction:

Active EAE was induced on Day 1 by the subcutaneous injection in the flanks at two injection sites, the encephalitogenic mixture (emulsion) consisting of MOG and commercial CFA containing 5 mg/mL Mycobacterium tuberculosis (MT) at a total volume of 0.2 mL/mouse in the right flank of the animals. Pertussis toxin was injected intra peritoneally on the day of induction and 48 hours later at dose level of 150 ng/0.2 ml/mouse. The dose of the MOG and MT is 150 μg/mouse and 500 μg/mouse respectively.

Study Design:

The mice were allocated randomly into 6 groups according to Table 2 below.

| | Treatment | | | Administration | Admin. |
|---|---|---|---|---|---|
| Group | groups | Dose/day | | Route | Period |
| 1 | Vehicle | 0.2 | ml/mouse | Gavage bid | From Day |
| | (05%MC) | | | (AM/PM) | 1 to 30 |
| 2 | 0.5% MC | 0.2 | ml/mouse | Gavage qd | From Day |
| | | | | (AM) | 1 to 30 |
| | LAQUINIMOD | 1 | mg/kg/day | Gavage qd | |
| | | | | (PM) | |
| 3 | 0.5% MC | 0.2 | ml/mouse | Gavage qd | From Day |
| | | | | (AM) | 1 to 30 |
| | LAQUINIMOD | 5 | mg/kg/day | Gavage qd | |
| | | | | (PM) | |
| 4 | Flupirtine | 30 | mg/kg/day | Gavage qd | From Day |
| | | | | (AM) | 1 to 30 |
| | 0.5% MC | 0.2 | ml/mouse | Gavage qd | |
| | | | | (PM) | |
| 5 | Flupirtine | 30 | mg/kg/day | Gavage qd | From Day |
| | | | | (AM) | 1 to 30 |
| | LAQUINIMOD | 1 | mg/kg/day | Gavage qd | |
| | | | | (PM) | |
| 6 | Flupirtine | 30 | mg/kg/day | Gavage qd | From Day |
| | | | | (AM) | 1 to 30 |
| | LAQUINIMOD | 5 | mg/kg/day | Gavage qd | |
| | | | | (PM) | |

### Preparation and Administration of Encephalitogenic Emulsion:

Oil portion: CFA (containing 1 mg/ml MT) was enriched with mycobacterium tuberculosis to yield 5 mg/ml MT.

Liquid portion: 20 mg MOG or equivalent was dissolved in 13.3 ml Normal saline to yield 1.5 mg/ml MOG.

Emulsion: The emulsions was made from equal parts of oil (13.3 mL CFA containing 5.0 mg/ml MT) and liquid portions (13.3 ml of 1.5 mg MOG/ml) in two syringes connected to each other with Leur lock to yield 0.75 mg/ml MOG. The emulsion was administered subcutaneous at two injection sites in the flanks of the mice in the study once on Day 1 of the study. The dose of the MOG in all the groups was 0.15 mg/0.2 ml/mouse.

The dose of the MT in all the groups was 0.5 mg/0.2 ml/mouse.

### Preparation and Administration of Pertussis Toxin:

75.0 μl Pertussis toxin (200 μg/ml) was added to 19.925 ml PBS to yield 0.750 μg/ml. 0.2 ml of 0.750 μg/ml.

Pertussis toxin solution was injected intraperitoneally immediately after MOG emulsion injection for dose level of 150 ng/mouse. Injection of the pertussis toxin was repeated in a similar manner after 48 hours.

### Preparation and Administration of Test Articles

### Flupirtine Formulations:

Formulation of flupirtine were prepared daily in 0.5% Methyl-cellulose/H₂O. A concentration of 3 mg/ml flupirtine-maleate was prepared for dose level of 30 mg/kg qd (groups #4, 5 and 6).

The mice were administered with flupirtine at volume dose level of 200 μl/mouse by the oral gavage route.

### Laquinimod Formulations:

Laquinimod was diluted in 0.5% methyl cellulose/H₂O.

For dose level of 5.0 mg/kg laquinimod, 0.5 mg/ml stock solution was prepared (groups #3 and 6).

For dose level of 1.0 mg/kg laquinimod, 0.1 mg/ml stock solution was prepared (groups #2 and 5).

Laquinimod was administered to the respective groups daily, by oral gavage at a volume of 200 ul/mouse. The test formulations were stored at 2 to 8° C. until use in amber colored bottles.

### Treatments:

The mice of respective groups were administered with laquinimod, flupirtine or the vehicle (0.5% Methyl cellulose) a daily dose, bolus qd by oral gavage at a volume of 200 μl/mouse.

All of the treatment groups were administered from Day 1, with the respective test formulations.

EAE CLINICAL SIGNS: The mice were observed daily from the 8th day post-EAE induction (first injection of MOG) and the EAE clinical signs were scored according to the grades described in the table presented below.

TABLE 3
Evaluation of the EAE clinical signs

| Score | Signs | Description |
|---|---|---|
| 0 | Normal behavior | No neurological signs. |
| 1 | Limp tail | Part or the whole tail is limp and droopy. |
| 2 | righting reflex | Animal has difficulties rolling onto his feet |
| | | when laid on its back |
| 3 | Hind leg | wobbly walk - when the mouse walks the hind |
| | weakness | legs are unsteady |
| 4 | Hind leg paralysis | The mouse drags its hind legs but is able to |
| | | move around using its fore legs |
| 5 | Full paralysis | The mouse can't move around, it looks thinner |
| | | and emaciated. |
| 6 | Moribund/Death | |

All mice with score 1 and above were considered sick. When the first clinical sign appears all mice were given food soaked in water, which was spread on different places on the bedding of the cages.

### Interpretation of Results Calculation of the Incidence of Disease (Disease Ratio)

The number of sick animals in each group were summed.
   The incidence of disease was calculated as

$$INCIDENCE of DISEASE = ( \frac{No . of sick mice in treated group}{No . of sick mice in control group} )$$

The percent inhibition according to incidence was calculated as

$$INHIBITION ( \% ) of INCIDENCE = ( 1 - \frac{Number of sick mice in treated group}{Number of sick mice in control group} ) \times 100$$

### Calculation of the Mortality/Moribundity Rate (Mortality Ratio)

The number of dead or moribund animals in each group were summed.
The mortality of disease was calculated as

$$MORTALITY of DISEASE = ( \frac{No . of dead or moribound mice in treated group}{No . of dead or moribound mice in control group} )$$

The percent inhibition according to mortality was calculated as

$$INHIBITION ( \% ) of MORTALITY = ( 1 - \frac{Number of dead or moribound mice in treated group}{Number of dead or moribound mice in control group} ) \times 100$$

### Calculation of Duration of Disease

The mean duration of disease expressed in days was calculated as

$$Mean Duration = ( \frac{Σ Duration of disease of each mouse}{No . of mice in the group} )$$

### Calculation of Mean Delay in Onset of Disease

The mean onset of disease expressed in days was calculated as

$$Mean Onset = ( \frac{Σ Onset of disease of each mouse}{No . of mice in the group} )$$

The mean delay in onset of disease expressed in days was calculated by subtracting the mean onset of disease in control group from test group.

### Calculation of the Mean Maximal Score and Percent Inhibition

The mean maximal score (MMS) of each group was calculated as

$$M M S = ( \frac{Σ Maximal Score of each mouse}{No . of mice in the group} )$$

The percent inhibition according to MMS was calculated as MMS of treated group

$$INHIBITION ( \% ) of M M S = ( 1 - \frac{M M S of treated group}{M M S of control group} ) \times 100$$

### Calculation of the Group Mean Score and Percent Inhibition

The daily scores of each mouse in the test group were summed and the individual mean daily score (IMS) was calculated as

$$I M S = ( \frac{Σ Daily score of mouse}{Observation period ( days )} )$$

The mean group score (GMS) was calculated as

$$G M S = ( \frac{Σ I M S of each mouse}{No . of mice in the group} )$$

The percent inhibition was calculated as

$$INHIBITION ( \% ) of G M S = ( 1 - \frac{G M S of treated group}{G M S of control group} ) \times 100$$

### Results

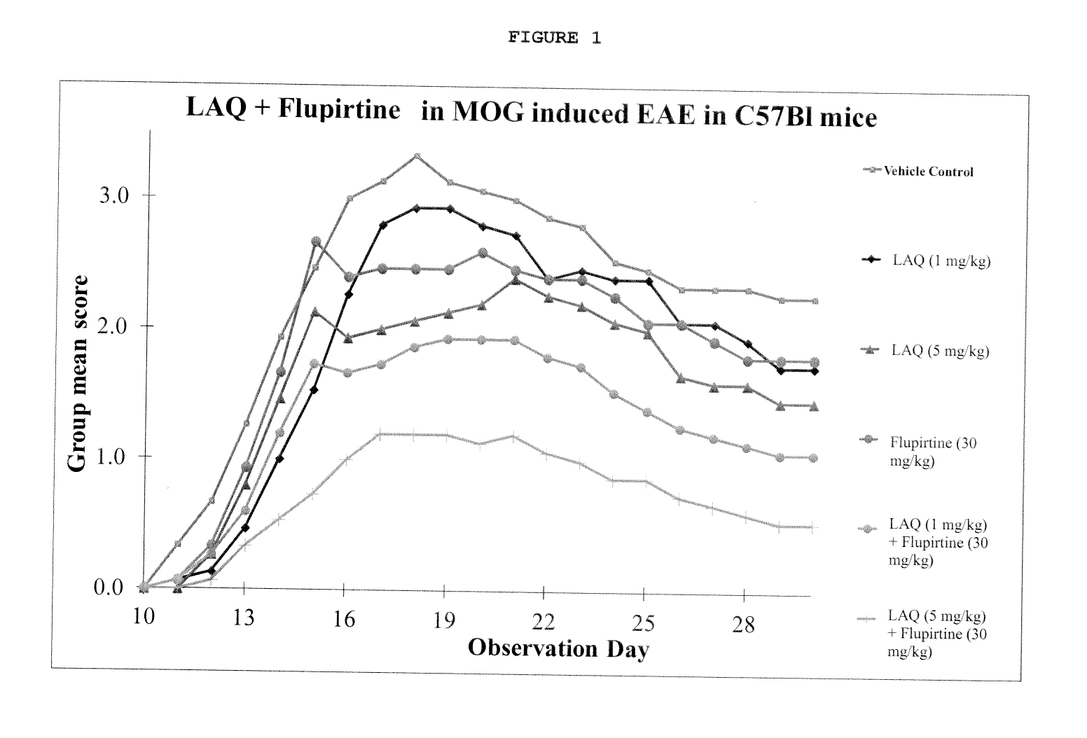

A summary of the incidence, mortality, Group Mean Score (GMS), duration of the disease, onset of the disease and the activity of each group compared to the vehicle treated control group is shown in the Summarized Table 4. The Clinical profile of the treatment groups are presented graphically in FIG. 1.

In the group treated with flupirtine (30/mg/kg), 17.4% activity was observed according to GMS when compared to the vehicle administered control group.

In groups treated with laquinimod at dose levels of 1 mg/kg, and 5 mg/kg, 17.4 and 30.4% activity, respectively, was observed according to GMS when compared to the vehicle administered control group.

The total blocking of EAE in the groups treated with flupirtine (30 mg/kg) in combination with laquinimod (1 mg/kg and 5 mg/kg) exhibited an activity higher than the additive effect of each treatment alone.

TABLE 4
Test Article: laquinimod and flupirtine alone and in combination.

| | | | | MMS | | GMS | | Onset | Duration |
|---|---|---|---|---|---|---|---|---|---|
| Treatment | MORT | INC | INH 1 | value | INH 2 | value | INH 3 | (days) | (days) |
| Vehicle (0.5% | 0/15 | 15/15 | — | 3.5 ± | — | 2.3 ± | — | 12.7 ± 1.7 | 17.9 ± |
| MC) bid | | | | 0.6 | | 0.7 | | | 2.5 |
| LAQ (1 mg/kg) + | 0/15 | 15/15 | 0% | 3.4 ± | 2.9% | 1.9 ± | 17.4% | 14.8 ± | 16.0 ± |
| 0.5% MC | | | | 0.8 | p = 0.8 | 0.7 | p = 0.07 | 2.7 | 2.8 |
| | | | | | | | | p = 0.01 | p = 0.02 |
| LAQ (5 mg/kg) + | 0/15 | 14/15 | 6.7% | 2.9 ± | 17.1% | 1.6 ± | 30.4% | 15.0 ± | 14.7 ± |
| 0.5% MC | | | | 1.2 | p = 0.2 | 1.8 | p = | 4.9 | 5.2 |
| | | | | | | | 0.02 | p = 0.09 | p = 0.03 |
| 0.5% MC + | 0/15 | 15/15 | 0% | 3.1 ± | 11.4% | 1.9 ± | 17.4% | 13.3 ± | 17.0 ± |
| Flupirtin (30 mg/kg) | | | | 0.7 | P = 0.09 | 0.6 | P = 0.08 | 1.6 | 2.7 |
| | | | | | | | | p = 0.35 | p = 0.2 |
| LAQ (1 mg/kg) + | 0/15 | 12/15 | 20.0% | 2.1 ± | 40.0% | 1.3 ± | 43.5% | 17.3 ± | 13.1 ± |
| Flupirtin (30 mg/kg) | | | | 1.3 | p = | 0.9 | p = | 7.3 | 7.3 |
| | | | | | 0.001 | | 0.005 | p = 0.023 | p = 0.02 |
| LAQ (5 mg/kg) + | 0/15 | 12/15 | 20.0% | 1.5 ± | 57.1% | 0.7 ± | 69.6% | 18.5 ± | 10.3 ± |
| Flupirtin (30 mg/kg) | | | | 1.1 | p < | 0.6 | p < | 6.9 | 6.9 |
| | | | | | 0.001 | | 0.001 | p = 0.001 | p < 0.001 |

MORT: Mortality
INC: Incidence
INH: Inhibition

### Conclusions

In this study, total blocking of EAE in the groups treated with flupirtine in combination with laquinimod (1 mg/kg and 5 mg/kg) exhibited an activity higher than the additive effect of each treatment alone, showing a synergistic effect of the treatment of flupirtine in combination with laquinimod. This unexpected result provides evidence that such a combination can be used for therapeutic treatment of human MS and CIS patients.

### Example 2

### Assessment of Efficacy of Laquinimod as Add-on Therapy to Flupirtine in Multiple Sclerosis (MS) Patients

Periodic oral administration of laquinimod (p.o. 0.6 mg/day or 1.2 mg/day) as an add-on therapy for a human patient afflicted with a form of MS who is already receiving flupirtine (p.o. 400 mg/day) provides a clinically meaningful advantage and is more effective (provides at least an additive effect or more than an additive effect) in treating the patient than when flupirtine is administered alone (at the same dose).

Periodic oral administration flupirtine (p.o. 400 mg/day) as an add-on therapy for a human patient afflicted with a form of MS who is already receiving of laquinimod (p.o. 0.6 mg/day or 1.2 mg/day) provides a clinically meaningful advantage and is more effective (provides at least an additive effect or more than an additive effect) in treating the patient than when laquinimod is administered alone (at the same dose).

The add-on therapies also provides efficacy (provides at least an additive effect or more than an additive effect) in treating the patient without undue adverse side effects or affecting the safety of the treatment. As compared to when each agent is administered alone:
1. The add-on therapy is more effective (provides an additive effect or more than an additive effect) in reducing the decrease in brain volume (determined by the percent brain volume change (PBVC)), in multiple sclerosis patients.
2. The add-on therapy is more effective (provides an additive effect or more than an additive effect) in increasing the time to confirmed disease progression (CDP), in multiple sclerosis patients, where CDP is defined as a sustained increase in EDSS of 21 point from Baseline for at least 3 months. Progression cannot be confirmed during a relapse.
3. The add-on therapy is more effective (provides an additive effect or more than an additive effect) in reducing abnormalities observed in whole Brain MTR histogram, in multiple sclerosis patients.
4. The add-on therapy is more effective (provides an additive effect or more than an additive effect) in reducing the number of confirmed relapses and therefore the relapse rate, in multiple sclerosis patients.
5. The add-on therapy is also more effective (provides an additive effect or more than an additive effect) in reducing the accumulation of physical disability in multiple sclerosis patients, as measured by the time to confirmed progression of EDSS.
6. The add-on therapy is more effective (provides an additive effect or more than an additive effect) in reducing MRI-monitored disease activity in multiple sclerosis patients, as measured by the cumulative number of T1 Gd-enhancing lesions on T1-weighted images, the cumulative number new T1 hypointense lesions, the cumulative number of new T2 lesions, the cumulative number of new T1 hypointense lesions on T1-weight images (black holes), the number of active (new T2 or GdE-T1) lesions, presence or absence of GdE lesions, change in total volume of T1 Gd-enhancing lesions, change in total volume of T2 lesions, and/or cortical thickness.
7. The add-on therapy is more effective (provides an additive effect or more than an additive effect) in reducing brain atrophy in multiple sclerosis patients.
8. The add-on therapy is more effective (provides an additive effect or more than an additive effect) in reducing the frequency of relapses, the frequency of clinical exacerbation, and the risk for confirmed progression in multiple sclerosis patients.
9. The add-on therapy is more effective (provides an additive effect or more than an additive effect) in increasing the time to confirmed relapse in multiple sclerosis patients.
10. The add-on therapy is more effective (provides an additive effect or more than an additive effect) in improving the general health status (as assessed by the EuroQoL (EQ5D) questionnaire), symptom severity on work (as assessed by the work productivity and activities impairment General Health (WPAI-GH) questionnaire) and quality of life, in multiple sclerosis patients.
11. The add-on therapy is more effective (provides an additive effect or more than an additive effect) in decreasing cerebral dysfunction/cognitive impairment (as assessed by Symbol Digit Modalities Test (SDMT)), in multiple sclerosis patients during the double blind study period.

Administration of laquinimod (p.o., 0.6 mg/day and 1.2 mg/day) as an add-on therapy to flupirtine (p.o., 400 mg/day) provides a clinically meaningful advantage and is more effective (provides an additive effect or more than an additive effect) in delaying the conversion to clinically definite MS in patients presenting a CIS suggestive of MS than when flupirtine is administered alone (at the same dose).

Administration of laquinimod (p.o., 0.6 mg/day and 1.2 mg/day) as an add-on therapy to flupirtine (p.o., 400 mg/day) provides a clinically meaningful advantage and is more effective (provides an additive effect or more than an additive effect) in reducing the rate of development of clinically definite MS, the occurrence of new MRI-detected lesions in the brain, the accumulation of lesion area in the brain and brain atrophy in persons at high risk for developing MS, and is more effective in reducing the occurrence of clinically definite MS and preventing irreversible brain damage in these persons than when flupirtine is administered alone (at the same dose).

Administration of flupirtine (p.o., 400 mg/day) as an add-on therapy to laquinimod (p.o., 0.6 mg/day and 1.2 mg/day) provides a clinically meaningful advantage and is more effective (provides an additive effect or more than an additive effect) in delaying the conversion to clinically definite MS in patients presenting a CIS suggestive of MS than when laquinimod is administered alone (at the same dose).

Administration of flupirtine (p.o., 400 mg/day) as an add-on therapy to laquinimod (p.o., 0.6 mg/day and 1.2 mg/day) provides a clinically meaningful advantage and is more effective (provides an additive effect or more than an additive effect) in reducing the rate of development of clinically definite MS, the occurrence of new MRI-detected lesions in the brain, the accumulation of lesion area in the brain and brain atrophy in persons at high risk for developing MS, and is more effective in reducing the occurrence of clinically definite MS and preventing irreversible brain damage in these persons than when laquinimod is administered alone (at the same dose).

### Example 3

### Assessment of Efficacy of Laquinimod in Combination with Flupirtine in Multiple Sclerosis (MS) Patients

Periodic oral administration of laquinimod (0.6 mg/day or 1.2 mg/day) in combination with flupirtine (p.o., 400 mg/day) to a human patient afflicted with relapsing form of multiple sclerosis provides increased efficacy (provides at least an additive effect or more than an additive effect) in treating the patient than when laquinimod is administered alone or when flupirtine is administered alone (at the same dose). The combination therapy also provides efficacy (provides at least an additive effect or more than an additive effect) in treating the patient without undue adverse side effects or affecting the safety of the treatment.

The combination therapy provides a clinically meaningful advantage and is more effective (provides at least an additive effect or more than an additive effect) in treating the patient than when laquinimod or flupirtine is administered alone (at the same dose) in the following manner:
1. The combination therapy is more effective (provides an additive effect or more than an additive effect) in reducing the decrease in brain volume (determined by the percent brain volume change (PBVC)), in multiple sclerosis patients.
2. The combination therapy is more effective (provides an additive effect or more than an additive effect) in increasing the time to confirmed disease progression (CDP), in multiple sclerosis patients, where CDP is defined as a sustained increase in EDSS of ≦1 point from Baseline for at least 3 months. Progression cannot be confirmed during a relapse.
3. The combination therapy is more effective (provides an additive effect or more than an additive effect) in reducing abnormalities observed in whole Brain MTR histogram, in multiple sclerosis patients during.
4. The combination therapy is more effective (provides an additive effect or more than an additive effect) in reducing the number of confirmed relapses and therefore the relapse rate, in multiple sclerosis patients.
5. The combination therapy is also more effective (provides an additive effect or more than an additive effect) in reducing the accumulation of physical disability in multiple sclerosis patients, as measured by the time to confirmed progression of EDSS.
6. The combination therapy is more effective (provides an additive effect or more than an additive effect) in reducing MRI-monitored disease activity in multiple sclerosis patients, as measured by the cumulative number of T1 Gd-enhancing lesions on T1-weighted images, the cumulative number new T1 hypointense lesions, the cumulative number of new T2 lesions, the cumulative number of new T1 hypointense lesions on T1-weight images (black holes), the number of active (new T2 or GdE-T1) lesions, presence or absence of GdE lesions, change in total volume of T1 Gd-enhancing lesions, change in total volume of T2 lesions, and/or cortical thickness.
7. The combination therapy is more effective (provides an additive effect or more than an additive effect) in reducing brain atrophy in multiple sclerosis patients.
8. The combination therapy is more effective (provides an additive effect or more than an additive effect) in reducing the frequency of relapses, the frequency of clinical exacerbation, and the risk for confirmed progression in multiple sclerosis patients.
9. The combination therapy is more effective (provides an additive effect or more than an additive effect) in increasing the time to confirmed relapse in multiple sclerosis patients.
10. The combination therapy is more effective (provides an additive effect or more than an additive effect) in improving the general health status (as assessed by the EuroQoL (EQ5D) questionnaire), symptom severity on work (as assessed by the work productivity and activities impairment General Health (WPAI-GH) questionnaire) and quality of life, in multiple sclerosis patients.
11. The combination therapy is more effective (provides an additive effect or more than an additive effect) in decreasing cerebral dysfunction/cognitive impairment (as assessed by Symbol Digit Modalities Test (SDMT)), in multiple sclerosis patients during the double blind study period.

Administration of laquinimod (p.o., 0.6 mg/day and 1.2 mg/day) in combination with flupirtine (p.o., 400 mg/day) provides a clinically meaningful advantage and is more effective (provides an additive effect or more than an additive effect) in delaying the conversion to clinically definite MS in patients presenting a CIS suggestive of MS than when flupirtine is administered alone (at the same dose).

Administration of laquinimod (p.o., 0.6 mg/day and 1.2 mg/day) in combination with flupirtine (p.o., 400 mg/day) provides a clinically meaningful advantage and is more effective (provides an additive effect or more than an additive effect) in reducing the rate of development of clinically definite MS, the occurrence of new MRI-detected lesions in the brain, the accumulation of lesion area in the brain and brain atrophy in persons at high risk for developing MS, and is more effective in reducing the occurrence of clinically definite MS and preventing irreversible brain damage in these persons than when flupirtine is administered alone (at the same dose).

### REFERENCES

1. “COPAXONE®” in Physician's Desk Reference, Thompson Reuters—Physician's Desk Reference Inc., Montvale, N. J., 2008, 3110-3113.
2. Barkhof, F. (1999) “MRI in Multiple Sclerosis: Correlation with Expanded Disability Status Scale (EDSS)”, Multiple Sclerosis. 5(4):283-286 (Abstract).
3. Bjartmar C and Fox R I. (2002) “Pathological mechanisms and disease progression of multiple sclerosis: therapeutic implication”, Drugs of Today. 38:7-29.
4. Brex et al., (2002) “A longitudinal study of abnormalities on MRI and disability from multiple sclerosis”, N Engl J Med. Jan. 17, 2002 346(3):158-64.
5. Brod et al. (2000) Annals of Neurology, 47:127-131.
6. Brück (2011) “Insight into the mechanism of laquinimod action.” J Neurol Sci. 2011 Jul. 15; 306(1-2):173-9.
7. Brunmark C et al., (2002) “The new orally active immunoregulator laquinimod (ABR-215062) effectively inhibits development and relapses of experimental autoimmune encephalomyelitis”, J Neuroimmunology. 130:163-172.
8. Chabot and Yong, Interferon-1b increases IL-10 in a model of T cell-microglia interaction: Relevance to MS, Neurol. 2000, 55: 1497-1505.
9. Chabot et al., Cytokine production in T lymphocyte-microglia interaction is attenuated by glatiramer acetate: A mechanism for therapeutic efficacy in multiple sclerosis, Mult. Scler., in press.
10. Clinical Trials Website, article entitled “Flupirtine as Oral Treatment in Multiple Sclerosis (FLORIMS)” retrieved on Jul. 23, 2013 from ClinicalTrials.gov, <http://clinicaltrials.gov/ct2/show/NCT00623415>.
11. Comi et al. (2007) LAQ/5062 Study Group. “The Effect of Two Doses of Laquinimod on MRI-Monitored Disease Activity in Patients with Relapsing-Remitting Multiple Sclerosis: A Multi-Center, Randomized, Double-Blind, Placebo-Controlled Study”, Presented at: 59th Annual Meeting of the American Academy of Neurology; April 28-May 5, 2007; Boston, Mass.
12. Compston, Genetic susceptibility to multiple sclerosis, in McAlpine's Multiple Sclerosis, Matthews, B. ed., London: Churchill Livingstone, 1991, 301-319.
13. Conway and Cohen (2010) “Combination therapy in multiple sclerosis”, Lancet Neurol, 9:299-308.
14. Costello et al. (2007) “Combination therapies for multiple sclerosis: scientific rationale, clinical trials, and clinical practice”, Current Opinion in Neurology, 20:281-285.
15. Dal Canto et al. (1977) Multiple sclerosis. Animal model: Theiler's virus infection in mice. Am. J. Path. 88:497-500.
16. DE 100 48 969 A1, published Mar. 14, 2002 (Müller-Schwefe).
17. DE 17 95 858 C2, granted Jan. 11, 1979 (Deutsche Gold- and Silber-Scheideanstalt vormals Roessler).
18. DE 195 41 405 A1, published May 15, 1997 (Pergande Gabriela et al.).
19. DE 31 33 519 C2, granted Feb. 9, 1989 (Bebenburg Walter von et al.).
20. DE 34 16 609 A1, published Nov. 7, 1985 (Hettche Helmut et al.).
21. DE 41 22 166 A1, published Jan. 16, 1992 (Lobisch Michael et al.).
22. DE 43 19 649 A1, published Sep. 22, 1994 (Goede Joachim et al.).
23. DE 93 21 574 U1, registered Jun. 29, 2000, date of registration published Aug. 3, 2000 (VIATRIS GmbH & Co. KG).
24. De Stefano et al. (1999) “Evidence of early axonal damage in patients with multiple sclerosis”, Neurology. 52(Suppl 2):A378.
25. Dunitz, M. Multiple sclerosis therapeutics, Ed. Rudick and Goodkin. London: Taylor & Francis, 1999.
26. Durelli et al. and the Independent Comparison of Interferon (INCOMIN) Trial Study Group. (2002) “Every-other-day interferon beta-1b versus once-weekly interferon beta-1a for multiple sclerosis: results of a 2-year prospective randomised multicentre study (INCOMIN)”, Lancet. 359:1453-60.
27. EMEA Guideline on Clinical Investigation of Medicinal Products for the Treatment of Multiple Sclerosis (CPMP/EWP/561/98 Rev. 1, November. 2006).
28. EPAR, Rebif®, Scientific Discussion.
29. Fernandez (2007) “Combination therapy in multiple sclerosis”, Journal of the neurological sciences, 259:95-103.
30. Filippi et al., Glatiramer acetate reduces the proportion of MS lesions evolving into black holes, Neurol., 2001, 57:731-733.
31. Fischer et al., (1999) “The Multiple Sclerosis Functional Composite measure (MSFC): an integrated approach to MS clinical outcome assessment” Multiple Sclerosis, 5(4):244-250.
32. Fisk et al., (1994) “Measuring the Functional Impact of Fatigue: Initial Validation of Fatigue Impact Scale”, Clin Inf Dis. 18 Suppl 1:S79-83.
33. Fisk et al., (1994) “The Impact of Fatigue on Patients with Multiple Sclerosis”, Can J Neurol Sci. 21:9-14.
34. Frenandez (2007) “Combination therapy in multiple sclerosis”, Journal of the neurological sciences, 259:95-103.
35. Frohman et al., (2003) “The utility of MRI in suspected MS: report of the Therapeutics and Technology Assessment Subcommittee of the American Academy of Neurology”, Neurology. Sep. 9, 2003, 61(5):602-11.
36. Gold (2008) “Combination therapies in multiple sclerosis”, J Neurol, 255[Suppl 1]:51-60.
37. Golder W, (2007) “Magnetic resonance spectroscopy in clinical oncology”, Onkologie. 27(3): 304-9.
38. Grossman et al., (1994) Magnetization transfer: theory and clinical applications in neuroradiology”, RadioGraphics. 14:279-290.
39. Guidance for Industry. In vivo drug metabolism/drug interaction studies—study design, data analysis, and recommendations for dosing and labeling, U.S. Dept. Health and Human Svcs., FDA, Ctr. for Drug Eval. and Res., Ctr. For Biologics Eval. and Res., Clin./Pharm., November 1999 <http://www.fda.gov/cber/gdlns/metabol.pdf>.
40. Gurevich et al. (2010) “Laquinimod suppress antigen presentation in relapsing-remitting multiple sclerosis: in vitro high-throughput gene expression study” (J Neuroimmunol. 2010 Apr. 15; 221(1-2):87-94. Epub 2010 Mar. 27.
41. Guyatt et al. (1985) “The 6-minute walk: a new measure of exercise capacity in patients with chronic heart failure”, Can Med Assoc J., 132:919-823.
42. Hafler and Weiner, M S: A CNS and systemic autoimmune disease, Immunol. Today, 1989, 10:104-107.
43. Hartung et al. (2005) “Significance of neutralizing antibodies to interferon beta during treatment of multiple sclerosis: expert opinions based on the Proceedings of an International Consensus Conference”, Eur J Neurol. 12:588-601.
44. Hauser et al. (1983) “Intensive immunosuppression in progressive multiple sclerosis”, New Engl J Med. 308:173-180.
45. Hohlfeld et al. (2000) “The neuroprotective effect of inflammation: implications for the therapy of multiple sclerosis”, J Neuroimmunol. 107:161-166.
46. Johnson et al., Copolymer 1 reduces relapse rate and improves disability in relapsing-remitting multiple sclerosis: results of a phase III multicenter, double-blind placebo-controlled trial. The Copolymer 1 Multiple Sclerosis Study Group, Neurol., 1995, 45:1268.
47. Kleinschmidt-DeMasters et al. (2005) New England Journal of Medicine, 353:369-379.
48. Kurtzke J F, (1983) “Rating neurologic impairment in multiple sclerosis: an expanded disability status scale (EDSS)”, Neurology 33(11):1444-1452.
49. Lampert, Autoimmune and virus-induced demyelinating diseases. A review, Am. J. Path., 1978, 91:176-208.
50. Langer-Gould et al. (2005) New England Journal of Medicine, 353:369-379.
51. Lublin F D, Reingold S C (1996) “Defining the clinical course of multiple sclerosis”, Neurol. 46:907-911.
52. Martyn, The epidemiology of multiple sclerosis, in McAlpine's Multiple Sclerosis, Matthews, B., ed., London: Churchil Livingstone, 1991, 3-40.
53. McDonald et al., Recommended diagnostic criteria for multiple sclerosis: guidelines from the International Panel on the diagnosis of multiple sclerosis. Ann. Neurol., 2001, 50:121-127.
54. McDonald, (2001) “Guidelines from the International Panel on the Diagnosis of Multiple Sclerosis” Ann. Neurol. 50:121-127.
55. Mehta et al. (1996) “Magnetization transfer magnetic resonance imaging: a clinical review”, Topics in Magnetic Resonance Imaging 8(4):214-30.
56. Miki et al. (1999) “Relapsing-Remitting Multiple Sclerosis: Longitudinal Analysis of MR Images—Lack of Correlation between Changes in T2 Lesion Volume and Clinical Findings”, Radiology. 213:395-399.
57. Milo and Panitch (2011) “Combination therapy in multiple sclerosis”, Journal of Neuroimmunology, 231(2011):23-31.
58. Moraal et al. (2008) “Subtraction MR Images in a Multiple Sclerosis Multicenter Clinical Trial Setting”, Radiology, 250(2):506-514.
59. Multiple sclerosis: its diagnosis, symptoms, types and stages, 2003 <http://www.albany.net/˜tjc/multiple-sclerosis.html>.
60. National MS Society Website, retrieved Jul. 10, 2012 <http://www.nationalmssociety.org/ms-clinical-care-network/researchers/clinical-study-measures/index.aspx>
61. Neuhaus et al. (2003) “Immunomodulation in multiple sclerosis: from immunosuppression to neuroprotection”, Trends Pharmacol Sci. 24:131-138.
62. Noseworthy et al. (2000) “Multiple sclerosis”, N Engl J Med. 343:938-952.
63. Olsson, Immunology of multiple sclerosis, Curr. Opin. Neurol. Neurosurg., 1992, 5:195-202.
64. Panitch et al. for the EVIDENCE (Evidence of Interferon Dose-response: European North American Comparative Efficacy) Study Group and the University of British Columbia MS/MRI Research Group. (2002) “Randomized comparative study of interferon β-1a treatment regiments in MS”, The EVIDENCE Trial. Neurology. 59:1496-1506.
65. Parkman, Graft-versus-host Disease, Ann. Rev. Med., 1991, 42:189-197.
66. PCT International Application Publication No. WO 1998/30227, published Jul. 16, 1998.
67. PCT International Application Publication No. WO 2000/05250, published Feb. 3, 2000.
68. PCT International Application Publication No. WO 2000/18794, published Apr. 6, 2000.
69. PCT International Application Publication No. WO 2003/048735, published Jun. 12, 2003.
70. PCT International Application Publication No. WO 2004/103297, published Dec. 2, 2004.
71. PCT International Application Publication No. WO 2004/112754, published Dec. 29, 2004.
72. PCT International Application Publication No. WO 2006/016036, published Nov. 2, 2006.
73. PCT International Application Publication No. WO 2006/029393, published Mar. 16, 2006.
74. PCT International Application Publication No. WO 2006/029411, published Mar. 16, 2006.
75. PCT International Application Publication No. WO 2006/083608, published Aug. 10, 2006.
76. PCT International Application Publication No. WO 2006/089164, published Aug. 24, 2006.
77. PCT International Application Publication No. WO 2006/116602, published Nov. 2, 2006.
78. PCT International Application Publication No. WO 2007/047863, published Apr. 26, 2007.
79. PCT International Application Publication No. WO 2007/146248, published Dec. 21, 2007, international filing date Jun. 12, 2007.
80. PCT International Application Publication No. WO 2009/070298, published Jun. 4, 2009.
81. PCT International Application Publication No. WO 2011/008274, published Jan. 20, 2011.
82. PCT International Application Publication No. WO 2011/022063, published Feb. 24, 2011.
83. PCT International Application Publication No. WO 2011/157719, published Dec. 22, 2011 (Hoock et al.).
84. PCT International Application Publication No. WO 2012/051106, published Apr. 19, 2012.
85. Polman et al. (2011) “Diagnostic Criteria for Multiple Sclerosis: 2010 Revisions to the McDonald Criteria”, Ann Neural, 69:292-302.
86. Polman et al., (2005) “Diagnostic criteria for multiple sclerosis: 2005 revisions to the McDonald Criteria”, Annals of Neurology, Volume 58 Issue 6, Pages 840-846.
87. Polman et al., (2005) “Treatment with laquinimod reduces development of active MRI lesions in relapsing MS”, Neurology. 64:987-991.
88. Poser et al. (1983) “New Diagnostic Criteria for Multiple Sclerosis: Guidelines for Research Protocols”, Annals of Neurology, March 1983, 13(3):227-230.
89. Rodriguez et al. (1987) Theiler's murine encephalomyelitis: a model of demyelination and persistence of virus. Crit. Rev. Immunol., 7:325.
90. Rosen Y, (2007) “The Recent advances in magnetic resonance neurospectroscopy”, Neurotherapeutics. 27(3):330-45.
91. RTT News Article dated April 12, 11, entitled “Teva Pharma, Active Biotech Post Positive Laquinimod Phase 3 ALLEGRO Results”.
92. Rudick et al. (2006) New England Journal of Medicine, 354:911-923.
93. Rudick, R. (1999) “Disease-Modifying Drugs for Relapsing-Remitting Multiple Sclerosis and Future Directions for Multiple Sclerosis Therapeutics”, Neurotherpatueics. 56:1079-1084.
94. Runström et al. (2002) “Laquinimod (ABR-215062) a candidate drug for treatment of Multiple Sclerosis inhibits the development of experimental autoimmune encephalomyelitis in IFN-β knock-out mice”, (Abstract), Medicon Valley Academy, Malmoe, Sweden.
95. Runström et al. (2006) “Inhibition of the development of chronic experimental autoimmune encephalomyelitis by laquinimod (ABR-215062) in IFN-β k.o. and wild type mice” Journal of Neuroimmunology, 173(2006):69-78.
96. Salama et al. (2003) Multiple Sclerosis, 9:28-31.
97. Sandberg-Wollheim et al. (2005) “48-week open safety study with high-dose oral laquinimod in patients”, Mult Scler. 11:S154 (Abstract).
98. Sorenson P S. (2006) “Neutralising antibodies to interferon-β-measurement, clinical relevance, and management”, J Neurol. 253[Suppl 6]:VI/16-VI/22.
99. Teitelbaum et al., Suppression of Experimental Allergic Encephalomyelitis by a Synthetic Polypeptide, Eur. J. Immunol., 1971, 1: 242-248.
100. Teitelbaum et al., Suppression of Experimental Allergic Encephalomyelitis with Basic Polymers, Eur. J. Immunol., 1973, 3: 273-279.
101. Teva Press Release dated Aug. 1, 2011, entitled “Results of Phase III BRAVO Trial Reinforce Unique Profile of Laquinimod for Multiple Sclerosis Treatment”.
102. The National MS Society (USA), The Disease Modifying Drug Brochure, Oct. 19, 2006.
103. U.S. Application Publication No. 2008-0279930, published Nov. 13, 2008 (Terhaag et al.).
104. U.S. Patent Application Publication No. 2008-0207526, published Aug. 28, 2008 (Strominger et al.).
105. U.S. Patent Application Publication No. 2008-0279952, published Nov. 13, 2008 (Bernd Terhaag and Pirna).
106. U.S. Patent Application Publication No. 2010-0322900, published Dec. 23, 2010 (Nora Tarcic and Modiin).
107. U.S. Patent Application Publication No. 2011-0027219, published Feb. 3, 2011 (Tarcic et al.).
108. U.S. Patent Application Publication No. 2011-0034508, published Feb. 10, 2011 (Liat Hayardeny).
109. U.S. Patent Application Publication No. 2011-0217295, published Sep. 8, 2011 (Haviv and Tarcic).
110. U.S. Patent Application Publication No. 2011-0218179, published Sep. 8, 2011 (Haviv and Tarcic).
111. U.S. Patent Application Publication No. 2011-0218203, published Sep. 8, 2011 (Joel Kaye et al.).
112. U.S. Patent Application Publication No. 2011-0230413, published Sep. 22, 2011 (Suhayl Dhib-Jalbut).
113. U.S. Patent Application Publication No. 2012-0010238, published Jan. 12, 2012 (Fristedt).
114. U.S. Patent Application Publication No. 2012-0010239, published Jan. 12, 2012 (Piryatinsky et al.).
115. U.S. Patent Application Publication No. 2012-0142730, published Jun. 7, 2012 (Tarcic et al.).
116. U.S. Pat. No. 3,849,550, issued Nov. 19, 1974 (Teitelbaum et al).
117. U.S. Pat. No. 5,284,861, issued Feb. 8, 1994 (Peter Emig et al.).
118. U.S. Pat. No. 5,721,258, issued Feb. 24, 1998 (Jurgen Engel et al.).
119. U.S. Pat. No. 5,800,808, issued Sep. 1, 1998 (Konfino et al).
120. U.S. Pat. No. 5,858,964, issued Jan. 12, 1999 (Aharoni et al).
121. U.S. Pat. No. 5,981,589, issued Nov. 9, 1999 (Konfino et al).
122. U.S. Pat. No. 6,048,898, issued Apr. 11, 2000 (Konfino et al).
123. U.S. Pat. No. 6,054,430, issued Apr. 25, 2000 (Konfino et al).
124. U.S. Pat. No. 6,077,851, issued Jun. 20, 2000 (Bjork et al).
125. U.S. Pat. No. 6,194,000, issued Feb. 27, 2001 (Smith et al.).
126. U.S. Pat. No. 6,214,791, issued Apr. 10, 2001 (Arnon et al).
127. U.S. Pat. No. 6,342,476, issued Jan. 29, 2002 (Konfino et al).
128. U.S. Pat. No. 6,362,161, issued Mar. 26, 2002 (Konfino et al).
129. U.S. Pat. No. 7,566,767, issued Jul. 28, 2009 (Strominger et al.).
130. U.S. Pat. No. 7,589,208, issued Sep. 15, 2009 (Jansson et al).
131. U.S. Pat. No. 7,884,208, issued Feb. 8, 2011 (Frenkel et al.).
132. U.S. Pat. No. 7,989,473, issued Aug. 2, 2011 (Patashnik et al.).
133. U.S. Pat. No. 8,008,258, issued Aug. 30, 2011 (Aharoni et al).
134. U.S. Pat. No. 8,178,127, issued May 15, 2012 (Safadi et al.).
135. Vollmer et al. (2008) “Glatiramer acetate after induction therapy with mitoxantrone in relapsing multiple sclerosis” Multiple Sclerosis, 00:1-8.
136. Yang et al., (2004) “Laquinimod (ABR-215062) suppresses the development of experimental autoimmune encephalomyelitis, modulates the Th1/Th2 balance and induces the Th3 cytokine TGF-β in Lewis rats”, J. Neuroimmunol. 156:3-9.
137. Yong (2002) “Differential mechanisms of action of interferon-β and glatiramer acetate in MS” Neurology, 59:1-7.
138. Zou et al. (2002) “Suppression of experimental autoimmune neuritis by ABR-215062 is associated with altered Th1/Th2 balance and inhibited migration of inflammatory cells into the peripheral nerve tissue”, Neuropharmacology. 42:731.

What is claimed is:

1. A method of treating a subject afflicted with multiple sclerosis or presenting a clinically isolated syndrome comprising administering to the subject an amount of laquinimod and an amount of flupirtine.

2. The method of claim 1, comprising periodically administering to the subject an amount of laquinimod and an amount of flupirtine, wherein the amounts when taken together are effective to treat the subject.

3. The method of claims 1 or 2, wherein the amount of laquinimod and the amount of flupirtine when administered together is more effective to treat the subject than when each agent at the same amount is administered alone.

4. The method of any one of claims 1-3, wherein the multiple sclerosis is relapsing multiple sclerosis.

5. The method of claim 4, wherein the relapsing multiple sclerosis is relapsing-remitting multiple sclerosis.

6. The method of any one of claims 1-5, wherein the amount of laquinimod and the amount of flupirtine when taken together is effective to reduce a symptom of multiple sclerosis in the subject.

7. The method of claim 6, wherein the symptom is a MRI-monitored multiple sclerosis disease activity, relapse rate, accumulation of physical disability, frequency of relapses, decreased time to confirmed disease progression, decreased time to confirmed relapse, frequency of clinical exacerbation, brain atrophy, neuronal dysfunction, neuronal injury, neuronal degeneration, neuronal apoptosis, risk for confirmed progression, deterioration of visual function, fatigue, impaired mobility, cognitive impairment, reduction of brain volume, abnormalities observed in whole Brain MTR histogram, deterioration in general health status, functional status, quality of life, and/or symptom severity on work.

8. The method of claim 7, wherein the amount of laquinimod and the amount of flupirtine when taken together is effective to decrease or inhibit reduction of brain volume.

9. The method of claim 8, wherein brain volume is measured by percent brain volume change (PBVC).

10. The method of claim 7, wherein the amount of laquinimod and the amount of flupirtine when taken together is effective to increase time to confirmed disease progression.

11. The method of claim 10, wherein time to confirmed disease progression is increased by 20-60%.

12. The method of claim 7, wherein the amount of laquinimod and the amount of flupirtine when taken together is effective to decrease abnormalities observed in whole Brain MTR histogram.

13. The method of claim 7, wherein the accumulation of physical disability is measured by Kurtzke Expanded Disability Status Scale (EDSS) score, or is assessed by the time to confirmed disease progression as measured by EDSS score.

14. The method of claim 13, wherein the subject had an EDSS score of 0-5.5 at baseline, an EDSS score of 1.5-4.5 at baseline or an EDSS score of 5.5 or greater at baseline.

15. The method of claims 13 or 14, wherein confirmed disease progression is a 1 point or a 0.5 point increase of the EDSS score.

16. The method of claim 7, wherein impaired mobility is assessed by the Timed-25 Foot Walk test, the 12-Item Multiple Sclerosis Walking Scale (MSWS-12) self-report questionnaire, the Ambulation Index (AI), the Six-Minute Walk (6 MW) Test or the Lower Extremity Manual Muscle Test (LEMMT) Test.

17. The method of claim 7, wherein the amount of laquinimod and the amount of flupirtine when taken together is effective to reduce cognitive impairment.

18. The method of claim 17, wherein cognitive impairment is assessed by the Symbol Digit Modalities Test (SDMT) score.

19. The method of claim 7, wherein general health status is assessed by the EuroQoL (EQ5D) questionnaire, Subject Global Impression (SGI) or Clinician Global Impression of Change (CGIC).

20. The method of claim 7, wherein functional status is measured by the subject's Short-Form General Health survey (SF-36) Subject Reported Questionnaire score.

21. The method of claim 7, wherein quality of life is assessed by SF-36, EQ5D, Subject Global Impression (SGI) or Clinician Global Impression of Change (CGIC).

22. The method of claims 20 or 21, wherein the subject's SF-36 mental component summary score (MSC) is improved.

23. The method of any one of claims 20-22, wherein the subject's SF-36 physical component summary sore (PSC) is improved.

24. The method of claim 7, wherein fatigue is assessed by the EQ5D, the subject's Modified Fatigue Impact Scale (MFIS) score or the French valid versions of the Fatigue Impact Scale (EMIF-SEP) score.

25. The method of claim 7, wherein symptom severity on work is measured by the work productivity and activities impairment General Health (WPAI-GH) questionnaire.

26. The method of any one of claims 1-25, wherein laquinimod is laquinimod sodium.

27. The method of any one of claims 1-26, wherein flupirtine is flupirtine maleate.

28. The method of any one of claims 1-27, wherein the laquinimod and/or the flupirtine is administered via oral administration.

29. The method of any one of claims 1-28, wherein flupirtine is administered in a slow release form or an immediate release form.

30. The method of any one of claims 1-27, wherein the laquinimod and/or the flupirtine is administered by injection.

31. The method of any one of claims 1-30, wherein the laquinimod and/or the flupirtine is administered daily.

32. The method of any one of claims 1-30, wherein the laquinimod and/or the flupirtine is administered more often than once daily.

33. The method of any one of claims 1-30, wherein the laquinimod and/or the flupirtine is administered less often than once daily.

34. The method of any one of claims 1-31, wherein the ratio by weight of the daily dose of flupirtine to laquinimod is in the range 142:1 to 2000:1.

35. The method of any one of claims 1-34, wherein the amount laquinimod administered is less than 0.6 mg/day.

36. The method of any one of claims 1-35, wherein the amount laquinimod administered is 0.1-40.0 mg/day.

37. The method of claim 36, wherein the amount laquinimod administered is 0.1-2.5 mg/day.

38. The method of claim 37, wherein the amount laquinimod administered is 0.25-2.0 mg/day.

39. The method of claim 38, wherein the amount laquinimod administered is 0.5-1.2 mg/day.

40. The method of claim 37, wherein the amount laquinimod administered is 0.25 mg/day, 0.3 mg/day, 0.5 mg/day, 0.6 mg/day, 1.0 mg/day, 1.2 mg/day, 1.5 mg/day or 2.0 mg/day.

41. The method of any one of claims 1-40, wherein the amount flupirtine administered is 10-1000 mg/day.

42. The method of claim 41, wherein the amount flupirtine administered is 50-500 mg/day.

43. The method of claim 42, wherein the amount flupirtine administered is 100-400 mg/day.

44. The method of claim 43, wherein the amount flupirtine administered is 400 mg/day.

45. The method of any one of claims 1-44, wherein a loading dose of an amount different form the intended dose is administered for a period of time at the start of the periodic administration.

46. The method of claim 45, wherein the loading dose is double the amount of the intended dose.

47. The method of any one of claims 1-46, wherein the subject is receiving laquinimod therapy prior to initiating flupirtine therapy.

48. The method claim 47, wherein the administration of laquinimod substantially precedes the administration of flupirtine.

49. The method of any one of claims 1-46, wherein the subject is receiving flupirtine therapy prior to initiating laquinimod therapy.

50. The method of claim 49, wherein the administration of flupirtine substantially precedes the administration of laquinimod.

51. The method of claim 49, where in the subject is receiving flupirtine therapy for at least 8 weeks, at least 10 weeks, at least 24 weeks, at least 28 weeks, at least 48 weeks or at least 52 weeks prior to initiating laquinimod therapy.

52. The method of any one of claims 1-51, further comprising administration of nonsteroidal anti-inflammatory drugs (NSAIDs), salicylates, slow-acting drugs, gold compounds, hydroxychloroquine, sulfasalazine, combinations of slow-acting drugs, corticosteroids, cytotoxic drugs, immunosuppressive drugs and/or antibodies.

53. The method of any one of claims 1-52, wherein the periodic administration of laquinimod and flupirtine continues for at least 3 days, for more than 30 days, for more than 42 days, for 8 weeks or more, for at least 12 weeks, for at least 24 weeks or for 6 months or more.

54. The method of any one of claims 1-53, wherein the administration of laquinimod and flupirtine inhibits a symptom of relapsing multiple sclerosis by at least 20%, by at least 30%, by at least 50%, by at least 70%, by more than 100%, by more than 300% or by more than 1000%.

55. The method of any one of claims 1-54, wherein each of the amount of laquinimod or pharmaceutically acceptable salt thereof when taken alone, and the amount of flupirtine when taken alone is effective to treat the subject.

56. The method of any one of claims 1-54, wherein either the amount of laquinimod or pharmaceutically acceptable salt thereof when taken alone, the amount of flupirtine when taken alone, or each such amount when taken alone is not effective to treat the subject.

57. The method of any one of claims 1-56, wherein the subject is a human patient.

58. A package comprising:
a) a first pharmaceutical composition comprising an amount of laquinimod and a pharmaceutically acceptable carrier;
b) a second pharmaceutical composition comprising an amount of flupirtine and a pharmaceutically acceptable carrier; and
c) instructions for use of the first and second pharmaceutical compositions together to treat a subject afflicted with multiple sclerosis or presenting a clinically isolated syndrome.

59. The package of claim 58, wherein the first pharmaceutical composition, the second pharmaceutical composition, or both the first and the second pharmaceutical composition are in an aerosol, an inhalable powder, an injectable, a liquid, a solid, a capsule or a tablet form.

60. The package of claim 58, wherein the first pharmaceutical composition, the second pharmaceutical composition, or both the first and the second pharmaceutical composition are in a liquid or a solid form.

61. The package of claim 60, wherein the first pharmaceutical composition, the second pharmaceutical composition, or both the first and the second pharmaceutical composition are in capsule form or in tablet form.

62. The package of claim 61, wherein the tablets are coated with a coating which inhibits oxygen from contacting the core.

63. The package of claim 62, wherein the coating comprises a cellulosic polymer, a detackifier, a gloss enhancer, or pigment.

64. The package of anyone of claims 58-63, wherein the first pharmaceutical composition further comprises mannitol.

65. The package of anyone of claims 58-64, wherein the first pharmaceutical composition further comprises an alkalinizing agent.

66. The package of claim 65, wherein the alkalinizing agent is meglumine.

67. The package of anyone of claims 58-66, wherein the first pharmaceutical composition further comprises an oxidation reducing agent.

68. The package of anyone of claims 58-64, wherein the first pharmaceutical composition is stable and free of an alkalinizing agent or an oxidation reducing agent.

69. The package of claim 68, wherein the first pharmaceutical composition is free of an alkalinizing agent and free of an oxidation reducing agent.

70. The package of anyone of claims 58-69, wherein the first pharmaceutical composition is stable and free of disintegrant.

71. The package of anyone of claims 58-70, wherein the first pharmaceutical composition further comprises a lubricant.

72. The package of claim 71, wherein the lubricant is present in the composition as solid particles.

73. The package of claims 71 or 72, wherein the lubricant is sodium stearyl fumarate or magnesium stearate.

74. The package of anyone of claims 58-73, wherein the first pharmaceutical composition further comprises a filler.

75. The package of claim 74, wherein the filler is present in the composition as solid particles.

76. The package of claims 74 or 75, wherein the filler is lactose, lactose monohydrate, starch, isomalt, mannitol, sodium starch glycolate, sorbitol, lactose spray dried, lactose anhydrouse, or a combination thereof.

77. The package of claim 76, wherein the filler is mannitol or lactose monohydrate.

78. The package of anyone of claims 58-77, further comprising a desiccant.

79. The package of claim 78, wherein the desiccant is silica gel.

80. The package of anyone of claims 58-79, wherein the first pharmaceutical composition is stable and has a moisture content of no more than 4%.

81. The package of anyone of claims 58-80, wherein laquinimod is present in the composition as solid particles.

82. The package of anyone of claims 58-81, wherein the package is a sealed packaging having a moisture permeability of not more than 15 mg/day per liter.

83. The package of claim 58-82, wherein the sealed package is a blister pack in which the maximum moisture permeability is no more than 0.005 mg/day.

84. The package of claim 83, wherein the sealed package is a bottle.

85. The package of claim 84, wherein the bottle is closed with a heat induction liner.

86. The package of anyone of claims 82-85, wherein the sealed package comprises an HDPE bottle.

87. The package of anyone of claims 82-86, wherein the sealed package comprises an oxygen absorbing agent.

88. The package of claim 87, wherein the oxygen absorbing agent is iron.

89. The package of any one of claims 58-88, wherein the amount of laquinimod in the first composition is less than 0.6 mg.

90. The package of any one of claims 58-89, wherein the amount of laquinimod in the first composition is 0.1-40.0 mg.

91. The package of claim 90, wherein the amount of laquinimod in the first composition is 0.1-2.5 mg.

92. The package of claim 91, wherein the amount of laquinimod in the first composition is 0.25-2.0 mg.

93. The package of claim 92, wherein the amount of laquinimod in the first composition is 0.5-1.2 mg.

94. The package of claim 91, wherein the amount of laquinimod in the first composition is 0.25 mg, 0.3 mg, 0.5 mg, 0.6 mg, 1.0 mg, 1.2 mg, 1.5 mg or 2.0 mg.

95. The package of any one of claim 58-94, wherein the amount of flupirtine is 10-1000 mg.

96. The package of claim 95 wherein the amount of flupirtine is 50-500 mg.

97. The package of claim 96, wherein the amount of flupirtine is 100-400 mg.

98. The package of claim 97, wherein the amount of flupirtine is 400 mg.

99. The package of any one of claims 58-98, wherein the amount of laquinimod and the amount of flupirtine are prepared to be administered simultaneously, contemporaneously or concomitantly.

100. Laquinimod for use as an add-on therapy or in combination with flupirtine in treating a subject afflicted with multiple sclerosis or presenting a clinically isolated syndrome.

101. Flupirtine for use as an add-on therapy or in combination with laquinimod in treating a subject afflicted with multiple sclerosis or presenting a clinically isolated syndrome.

102. A pharmaceutical composition comprising an amount of laquinimod and an amount of flupirtine for use in treating a subject afflicted with multiple sclerosis or presenting a clinically isolated syndrome, wherein the laquinimod and the flupirtine are prepared to be administered simultaneously, contemporaneously or concomitantly.

103. A pharmaceutical composition comprising an amount of laquinimod and an amount of flupirtine.

104. The pharmaceutical composition of claims 102 or 103, wherein the ratio of flupirtine to laquinimod by weight is in the range 142:1 to 2000:1.

105. The pharmaceutical composition of any one of claims 102-104, wherein laquinimod is laquinimod sodium.

106. The pharmaceutical composition of any one of claims 102-105, wherein flupirtine is flupirtine maleate.

107. The pharmaceutical composition of any one of claims 102-106, in an aerosol, an inhalable powder, an injectable, a liquid, a solid, a capsule or a tablet form.

108. The pharmaceutical composition of any one of claims 102-107, in a liquid or a solid form.

109. The pharmaceutical composition of claim 108, in capsule form or in tablet form.

110. The pharmaceutical composition of claim 109, wherein the tablets are coated with a coating which inhibits oxygen from contacting the core.

111. The pharmaceutical composition of claim 110, wherein the coating comprises a cellulosic polymer, a detackifier, a gloss enhancer, or pigment.

112. The pharmaceutical composition of anyone of claims 102-111, further comprising mannitol.

113. The pharmaceutical composition of anyone of claims 102-112, further comprising an alkalinizing agent.

114. The pharmaceutical composition of claim 113, wherein the alkalinizing agent is meglumine.

115. The pharmaceutical composition of anyone of claims 102-114, further comprising an oxidation reducing agent.

116. The pharmaceutical composition of anyone of claims 102-115, which is free of an alkalinizing agent or an oxidation reducing agent.

117. The pharmaceutical composition of claim 116, which is free of an alkalinizing agent and free of an oxidation reducing agent.

118. The pharmaceutical composition of anyone of claims 102-117, which is stable and free of disintegrant.

119. The pharmaceutical composition of anyone of claims 102-118, further comprising a lubricant.

120. The pharmaceutical composition of claim 119, wherein the lubricant is present in the composition as solid particles.

121. The pharmaceutical composition of claims 119 or 120, wherein the lubricant is sodium stearyl fumarate or magnesium stearate.

122. The pharmaceutical composition of anyone of claims 102-121, further comprising a filler.

123. The pharmaceutical composition of claim 122, wherein the filler is present in the composition as solid particles.

124. The pharmaceutical composition of claims 122 or 123, wherein the filler is lactose, lactose monohydrate, starch, isomalt, mannitol, sodium starch glycolate, sorbitol, lactose spray dried, lactose anhydrouse, or a combination thereof.

125. The pharmaceutical composition of claim 124, wherein the filler is mannitol or lactose monohydrate.

126. The pharmaceutical composition of any one of claims 102-125, wherein the amount of laquinimod in the composition is less than 0.6 mg.

127. The pharmaceutical composition of any one of claims 102-126, wherein the amount of laquinimod in the composition is 0.1-40.0 mg.

128. The pharmaceutical composition of claim 127, wherein the amount of laquinimod in the composition is 0.1-2.5 mg.

129. The pharmaceutical composition of claim 128, wherein the amount of laquinimod in the composition is 0.25-2.0 mg.

130. The pharmaceutical composition of claim 129, wherein the amount of laquinimod in the composition is 0.5-1.2 mg.

131. The pharmaceutical composition of claim 128, wherein the amount of laquinimod in the composition is 0.25 mg, 0.3 mg, 0.5 mg, 0.6 mg, 1.0 mg, 1.2 mg, 1.5 mg, 2.0 mg.

132. The pharmaceutical composition of any one of claims 102-131, wherein the amount of flupirtine in the composition is 10-1000 mg.

133. The pharmaceutical composition of claim 132, wherein the amount of flupirtine in the composition is 50-500 mg.

134. The pharmaceutical composition of claim 133, wherein the amount of flupirtine in the composition is 100-400 mg.

135. The pharmaceutical composition of claim 134, wherein the amount of flupirtine in the composition is 400 mg.

136. Use of an amount of laquinimod and an amount of flupirtine in the preparation of a combination for treating a subject afflicted with multiple sclerosis or presenting a clinically isolated syndrome wherein the laquinimod and the flupirtine are prepared to be administered simultaneously, contemporaneously or concomitantly.

137. A pharmaceutical composition comprising an amount of laquinimod for use in treating a subject afflicted with multiple sclerosis or presenting a clinically isolated syndrome as an add-on therapy or in combination with flupirtine.

138. A pharmaceutical composition comprising an amount of flupirtine for use treating a subject afflicted with multiple sclerosis or presenting a clinically isolated syndrome as an add-on therapy or in combination with laquinimod.

139. A pharmaceutical composition comprising an amount of laquinimod for use in treating a subject afflicted with multiple sclerosis or presenting a clinically isolated syndrome simultaneously, contemporaneously or concomitantly with flupirtine.

140. A pharmaceutical composition comprising an amount of flupirtine for use treating a subject afflicted with multiple sclerosis or presenting a clinically isolated syndrome simultaneously, contemporaneously or concomitantly with laquinimod.

141. A therapeutic package for dispensing to, or for use in dispensing to, a subject afflicted with multiple sclerosis or presenting a clinically isolated syndrome, which comprises:
a) one or more unit doses, each such unit dose comprising:
i) an amount of laquinimod and
ii) an amount of flupirtine
wherein the respective amounts of said laquinimod and said flupirtine in said unit dose are effective, upon concomitant administration to said subject, to treat the subject, and
b) a finished pharmaceutical container therefor, said container containing said unit dose or unit doses, said container further containing or comprising labeling directing the use of said package in the treatment of said subject.

142. The therapeutic package of claim 141, wherein the respective amounts of said laquinimod and said flupirtine in said unit dose when taken together is more effective to treat the subject than when compared to the administration of said laquinimod in the absence of said flupirtine or the administration of said flupirtine in the absence of said laquinimod.

143. A pharmaceutical composition in unit dosage form, useful in treating a subject afflicted with multiple sclerosis or presenting a clinically isolated syndrome, which comprises:
a) an amount of laquinimod;
b) an amount of flupirtine,
wherein the respective amounts of said laquinimod and said flupirtine in said composition are effective, upon concomitant administration to said subject of one or more of said unit dosage forms of said composition, to treat the subject.

144. The pharmaceutical composition of claim 143, wherein the respective amounts of said laquinimod and said flupirtine in said unit dose when taken together is more effective to treat the subject than when compared to the administration of said laquinimod in the absence of said flupirtine or the administration of said flupirtine in the absence of said laquinimod.
